SEQUENCE LISTING (10 sequences)

SEQ ID NO: 1 - protein, 8 aa, Mus musculus
YKFEVYEK

SEQ ID NO: 2 - protein, 9 aa, Mus musculus
KTWFSIPEK

SEQ ID NO: 3 - protein, 14 aa, Mus musculus
NGDLPWPPLRNEFK

SEQ ID NO: 4 - protein, 18 aa, Mus musculus
IMQEFESDTFFPEIDLGK

SEQ ID NO: 5 - protein, 23 aa, Mus musculus
GSGIMVRPLNSIVAVSQNMGIGK

SEQ ID NO: 6 - protein, 8 aa, Mus musculus
QNLVIMGR

SEQ ID NO: 7 - protein, 7 aa, Mus musculus
SLDDALR

SEQ ID NO: 8 - protein, 10 aa, Mus musculus
NGDLPWPPLR

SEQ ID NO: 9 - protein, 214 aa, Mus musculus
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 220 aa, Mus musculus
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

(19) United States
(12) Patent Application Publication
(10) Pub. No.: US 2009/0035836 A1
(43) Pub. Date: Feb. 5, 2009

(54) MODULATING PH-SENSITIVE BINDING USING NON-NATURAL AMINO ACIDS

(75) Inventors: Deepshikha Datta, South Pasadena, CA (US); William A. Goddard, Pasadena, CA (US); David Tirrell, Pasadena, CA (US); Joyce Yaochun Peng, Pasadena, CA (US)

Correspondence Address: SEED INTELLECTUAL PROPERTY LAW GROUP PLLC, 701 FIFTH AVE, SUITE 5400, SEATTLE, WA, 98104, US

(73) Assignee: CALIFORNIA INSTITUTE OF TECHNOLOGY, Pasadena, CA (US)

(21) Appl. No.: 12/147,379

(22) Filed: Jun. 26, 2008

Related U.S. Application Data
(62) Division of application No. 11/094,625, filed on Mar. 30, 2005, now abandoned.
(60) Provisional application No. 60/557,541, filed on Mar. 30, 2004.

(51) Int. Cl. C12N 7/00; C07K 2/00; C07K 16/00

(52) U.S. Cl. 435/235.1; 530/300; 530/387.1; 530/388.1; 530/387.3

(57) ABSTRACT

The invention provides methods, systems and reagents for regulating pH-sensitive protein interaction by incorporating non-natural amino acids into the protein (e.g. an antibody, or its functional fragment, derivative, etc.). The invention also relates to specific uses in regulating pH-sensitive binding of antibodies to tumor site, by conferring enhanced tumor-specificity/selectivity. In that embodiment, the non-natural amino acids preferably have desirable side-chain pKa's, such that at below physiological pH (e.g. about pH 6.3-6.5) the non-natural amino acid confer enhanced binding to tumor antigens in acidic environments. Such non-natural amino acids can be incorporated by any suitable means, such as by utilizing a modified aminoacyl-tRNA synthetase to charge the nonstandard amino acid to a modified tRNA, which forms strict Watson-Crick base-pairing with a codon that normally forms wobble base-pairing with natural tRNAs (e.g. the degenerate codon orthogonal system.

This application claims the benefit of the filing date of U.S. Provisional Application 60/557,541, filed on Mar. 30, 2004, the entire content of which is incorporated herein by reference.

### BACKGROUND OF THE INVENTION

### Background of the Invention

Many protein interactions are pH-sensitive, in the sense that binding affinity of one protein for its usual binding partner may change as environmental pH changes. For example, many ligands (such as insulin, interferons, growth hormone, etc.) bind their respective cell-surface receptors to elicit signal transduction. The ligand-receptor complex will then be internalized by receptor-mediated endocytosis, and go through a successive series of more and more acidic endosomes. Eventually, the ligand-receptor interaction is weakened at a certain acidic pH (e.g., about pH 5.0), and the ligand dissociates from the receptor. Some receptors (and perhaps some ligands) may be recycled back to cell surface. There, they may be able to bind their respective normal binding partners.

If the pH-sensitive binding can be modulated such that the ligand-receptor complex can be dissociated at a relatively higher pH, then certain ligands may be dissociated earlier from their receptors, and become preferentially recycled to cell surface rather than be degraded. This will result in an increased in vivo half-life of such ligands, which might be desirable since less insulin may be needed for the same (or better) efficacy in diabete patients.

In other situations, it might be desirable to modulate the pH-sensitive binding by favoring binding at a lower pH.

For example, monoclonal antibodies are generally very specific for their targets. However, in many applications, such as in cancer therapy, they tend to elicit certain side effects by, for example, binding to non-tumor tissues. One reason could be that the tumor targets against which monoclonal antibodies are raised are not specifically expressed on tumor cells, but are also expressed (although may be in smaller numbers) on some healthy cells. Such side effects are generally undesirable, and there is a need for antibodies with an improved specificity.

The pH of human blood is highly regulated and maintained in the range of about 7.6-7.8. On the other hand, tumor cells have an extracellular pH of 6.3-6.5, due to the accumulation of metabolic acids that are inefficiently cleared because of poor tumor vascularization. If the interaction between a tumor antigen and its therapeutic antibody can be modulated such that at low pH, the binding is favored, the tumor-antibody may have an added specificity/affinity/selectivity for those tumor antigens, even though the same tumor antigens are also occasionally found on normal tissues.

In fact, such modified antibodies may be desirable not only for cancer therapy, but also desirable for any antigen-antibody binding that may occur at a lower-than-normal level of pH.

Certainly, in the tumor antibody case, differences other than pH-sensitive binding in the extracellular region outside a tumor may also be explored to enhance tumor-specific binding. Such differences may include hypoxia condition and/or differences in the enzymes present in the extracellular environment of tumors relative to healthy tissues.

Tumor Hypoxia. Due to the increased metabolic needs of tumor cells and the fact that tumor growth exceeds that of its supporting vasculature, oxygen is often in short supply in or around tumor tissues. This leads to tumor hypoxia. Certain enzymes are expressed during hypoxia, which characteristics have been exploited to convert cancer prodrugs into active agents.

Tumor-Specific Extracellular Enzymes. Some tumor-specific enzymes that accumulate in the local extracellular tumor environment can also be investigated as prodrug activators.

While it has been known that there are differences in the micro-environment of tumors and non-tumor tissues, such differences have not been used to design and prepare antitumor antibodies with improved specificity.

Protein engineering is a powerful tool for modification of the structural catalytic and binding properties of natural proteins and for the de novo design of artificial proteins. Protein engineering relies on an efficient recognition mechanism for incorporating mutant amino acids in the desired protein sequences. Though this process has been very useful for designing new macromolecules with precise control of composition and architecture, a major limitation is that the mutagenesis is restricted to the 20 naturally occurring amino acids. However, it is becoming increasingly clear that incorporation of non-natural amino acids can extend the scope and impact of protein engineering methods. Thus, for many applications of designed macromolecules, it would be desirable to develop methods for incorporating amino acids that have novel chemical functionality not possessed by the 20 amino acids commonly found in naturally occurring proteins. That is, ideally, one would like to tailor changes in a protein (the size, acidity, nucleophilicity, hydrogen-bonding or hydrophobic properties, etc. of amino acids) to fulfill a specific structural or functional property of interest. The ability to incorporate such amino acid analogs into proteins would greatly expand our ability to rationally and systematically manipulate the structures of proteins, both to probe protein function and create proteins with new properties. For example, the ability to synthesize large quantities of proteins containing heavy atoms would facilitate protein structure determination, and the ability to site specifically substitute fluorophores or photo-cleavable groups into proteins in living cells would provide powerful tools for studying protein functions in vivo. One might also be able to enhance the properties of proteins by providing building blocks with new functional groups, such as an amino acid containing a keto-group.

Incorporation of novel amino acids in macromolecules has been successful to an extent. Biosynthetic assimilation of non-canonical amino acids into proteins has been achieved largely by exploiting the capacity of the wild type synthesis apparatus to utilize analogs of naturally occurring amino acids (Budisa 1995, Eur. J. Biochem 230: 788-796; Deming 1997, J. Macromol. Sci. Pure Appl. Chem. A34; 2143-2150; Duewel 1997, Biochemistry 36: 3404-3416; van Hest and Tirrell 1998, FEBS Lett 428(1-2): 68-70; Sharma et al., 2000, FEBS Lett 467(1): 37-40). Nevertheless, the number of amino acids shown conclusively to exhibit translational activity in vivo is small, and the chemical functionality that has been accessed by this method remains modest. In designing macromolecules with desired properties, this poses a limitation since such designs may require incorporation of complex analogs that differ significantly from the natural substrates in terms of both size and chemical properties and hence, are unable to circumvent the specificity of the synthetases. Thus, there is a need to develop a method to further expand the range of non-natural amino acids that can be incorporated.

In recent years, several laboratories have pursued an expansion in the number of genetically encoded amino acids, by using either a nonsense suppressor or a frame-shift suppressor tRNA to incorporate non-canonical amino acids into proteins in response to amber or four-base codons, respectively (Bain et al., J. Am. Chem. Soc. 111: 8013, 1989; Noren et al., Science 244: 182, 1989; Furter, Protein Sci. 7: 419, 1998; Wang et al., Proc. Natl. Acad. Sci. U.S.A., 100: 56, 2003; Hohsaka et al., FEBS Lett. 344:171:1994; Kowal and Oliver, Nucleic Acids Res. 25: 4685, 1997). Such methods insert non-canonical amino acids at codon positions that will normally terminate wild-type peptide synthesis (e.g. a stop codon or a frame-shift mutation). These methods have worked well for single-site insertion of novel amino acids. However, their utility in multisite incorporation is limited by modest (20-60%) suppression efficiencies (Anderson et al., J. Am. Chem. Soc. 124: 9674, 2002; Bain et al., Nature 356: 537, 1992; Hohsaka et al., Nucleic Acids Res. 29: 3646, 2001). This is so partially because too high a stop codon suppression efficiency will interfere with the normal translation termination of some non-targeted proteins in the organism. On the other hand, a low suppression efficiency will likely be insufficient to suppress more than one nonsense or frame-shift mutation sites in the target protein, such that it becomes more and more difficult or impractical to synthesize a full-length target protein incorporating more and more non-canonical amino acids.

Efficient multisite incorporation has been accomplished by replacement of natural amino acids in auxotrophic Escherichia coli strains, and by using aminoacyl-tRNA synthetases with relaxed substrate specificity or attenuated editing activity (Wilson and Hatfield, Biochim. Biophys. Acta 781: 205, 1984; Kast and Hennecke, J. Mol. Biol. 222: 99, 1991; Ibba et al., Biochemistry 33: 7107, 1994; Sharma et al., FEBS Lett. 467: 37, 2000; Tang and Tirrell, Biochemistry 41: 10635, 2002; Datta et al., J. Am. Chem. Soc. 124: 5652, 2002; Doring et al., Science 292: 501, 2001). Although this method provides efficient incorporation of analogues at multiple sites, it suffers from the limitation that the novel amino acid must “share” codons with one of the natural amino acids. Thus for any given codon position where both natural and novel amino acids can be inserted, other than a probability of incorporation, there is relatively little control over which amino acid will end up being inserted. This may be undesirable, since for an engineered enzyme or protein, non-canonical amino acid incorporation at an unintended site may unexpectedly compromise the function of the protein, while missing incorporating the non-canonical amino acid at the designed site will fail to achieve the design goal.

### SUMMARY OF THE INVENTION

One aspect of the invention provides a modified protein comprising one or more non-natural amino acid(s), the non-natural amino acid(s) confers or substantially alters pH-sensitive binding of the protein to its binding partner.

In one embodiment, the binding partner is a polypeptide, a nucleic acid, a polysaccharide, a lipid, a steroid, a polymer, a small molecule, or a metal ion.

In one embodiment, the modified protein is a modified antibody.

In one embodiment, the non-natural amino acid(s) confers or substantially alters pH-sensitive binding of the protein to its binding partner when the pH value changes at least about 0.5, 0.6, 0.7, 0.8, 0.9, 1.0, 1.5, 2.0, 3.0, 4.0, 5.0, 6.0, 7.0, 10 or more pH units.

In one embodiment, the non-natural amino acid(s) confers the modified antibody enhanced specifically, selectively, or affinity towards an antigen in a tissue at a specific pH.

In one embodiment, the specific pH is an extracellular pH at least about 0.5 unit higher or lower than a physiological pH.

In one embodiment, the specific pH is an extracellular pH at least about 1-1.5 units higher or lower than a physiological pH.

In one embodiment, the tissue is a neoplastic tissue, such as breast cancer overexpressing HER-2/neu.

In one embodiment, the tissue is undergoing a pathological condition selected from: tissue acidosis, inflammation, ischemia, infection, around tumors, fracture, hematoma, edema, blister, Tuberculosis abscess, a destructive inflammation state, arthritic, ulcer, or cystitis.

In one embodiment, the modified protein is a modified monoclonal antibody.

In one embodiment, the modified protein is a modified monoclonal antibody, or a functional fragment or derivative thereof selected from: Fab, Fab′, F(ab)₂, Fd, Fv, ScFv, diabody, tribody, tetrabody, dimer, trimer, or minibody.

In one embodiment, the modified protein is modified based on RITUXAN® (Rituximab), TIUXAN (Ibritumomab), BEXXAR® (Tositumomab and Iodine I131 Tositumomab), HERCEPTIN® (Trastuzumab), ZEVALIN® (Ibritumomab Tiuxetan), AVASTIN™ (Bevacizumab), ERBITUX™ (Cetuximab), MYLOTARG™ (Gemtuzumab-Ozogamicin for Injection), CAMPATH® (Alemtuzumab), PANOREX® (Edrecolomab), ZENAPAX® (Daclizumab), CeaVac (Anti-Idiotype (Anti-Id) Monoclonal Antibody (Mab)), IGN101 (murine mAb 17-1A), IGN311 (humanized monoclonal antibody), BEC2 (anti-idiotypic monoclonal antibody), IMC-1C11 (KDR receptor monoclonal antibody), LymphoCycle (Epratuzumab), or Pentumomab.

In one embodiment, the modified protein is modified by inserting the non-natural amino acid(s), or substituting one or more natural amino acid(s) in the antibody with the non-natural amino acid(s).

In one embodiment, the natural amino acid(s) is histidine. In another embodiment, the codon of a non-histidine natural amino acid(s) is changed to that of histidine for incorporation of the non-natural amino acid(s).

In one embodiment, the non-natural amino acid(s) comprises a side-chain that is not charged at pH of about 6.3-6.5.

In one embodiment, the pKa of the non-natural amino acid(s) is about 2.5-3.5 pH units lower than that of the natural amino acid(s).

In one embodiment, the non-natural amino acid(s) is selected from: 1,2,4-triazole-3-alanine, 2-fluoro-histidine, L-methyl histidine, 3-methyl-L-histidine, β-2-thienyl-L-alanine, or β-(2-Thiazolyl)-DL-alanine.

In one embodiment, the non-natural amino acid is a histidine analog with one or more substitutions on positions 2 and 4 of the histidine imidazole ring, by one or more of the groups selected from: —CN, —F, —Cl, —CH₂F, —OCH₃, or —CH₃.

The non-natural amino acid residue(s) can, in theory, be placed anywhere in the antibody structure. In one embodiment, the non-natural amino acid residue is placed in the Fab, for example, within the antibody variable region. In another embodiment, the non-natural amino acid residue is placed in the Fc-region. In another embodiment, the non-natural amino acid residue is placed in the binding interface of the antibody. In yet another embodiment, the non-natural amino acid residue is placed in the V_{H} region. In another embodiment, the substitution of natural amino acids by non-amino acids occur in any of these regions.

In one embodiment, the non-natural amino acid(s) confer enhanced binding affinity to Fc-receptor and/or to Clq of the complement system.

In a preferred embodiment, an antibody of the invention will have an altered (e.g. enhanced) affinity/specificity for an antigen or a protein binding partner (e.g., Clq of the complement and/or the Fc receptors on macrophages, etc.) in a tumor environment compared to a non-tumor environment.

In one embodiment, the natural amino acid(s) is present in the Fab-region of the antibody.

In one embodiment, the natural amino acid(s) is present in the V_{H} region of the antibody.

In one embodiment, the natural amino acid(s) is present in the binding interface of the antibody.

In one embodiment, the non-natural amino acid(s) is sterically similar to the natural amino acid(s).

In one embodiment, the non-natural amino acid(s) is sterically dissimilar to the natural amino acid(s).

In one embodiment, the modified protein further comprises mutated amino acid(s) adjacent to the non-natural amino acid(s) for maintaining binding affinity and/or specificity of the antibody.

In one embodiment, two or more natural amino acids in the antibody are substituted with at least two different non-natural amino acids.

In one embodiment, at least two or more natural amino acids in the antibody are substituted with the same non-natural amino acids.

In one embodiment, the antibody has an enhanced affinity for its target antigen in a tumor environment compared to a non-tumor environment.

In one embodiment, the modified protein has an enhanced affinity for the antigen in a tumor environment compared to a non-tumor environment.

In one embodiment, the non-natural amino acid(s) does not substantially alter the affinity/specificity of the modified antibody for the antigen.

In one embodiment, the non-natural amino acid(s) has a side-chain pKa between the pH at the tumor environment and the pH at the non-tumor environment.

The antibody primary sequence can be one of a known antibody that binds to a tumor antigen, or can be a sequence selected or designed to bind to a tumor antigen.

Another aspect of the invention provides a method to modify a protein to confer or substantially alter pH-sensitive binding to the protein, the method comprising: (1) inserting one or more non-natural amino acid(s) into the protein, or (2) replacing one or more natural amino acid(s) of the protein with the one or more non-natural amino acid(s), wherein the non-natural amino acid(s) confers or substantially alters pH-sensitive binding of the protein to a binding partner.

In one embodiment, the protein is an antibody, and the binding partner is present in a tumor tissue.

In one embodiment, an initial amino acid residue may be identified in the antibody sequence that is at the binding interface with the target protein. A mutant/variant protein can be prepared having a non-natural amino acid at the position of the initial amino acid. The non-natural amino acid could be sterically similar or dissimilar to the natural amino acid. If it is dissimilar, the amino acids in the proximity of the non-natural amino acid could be mutated to accept the new non-natural acid, and to maintain binding affinity and specificity for the target.

In a preferred embodiment, if more than one initial amino acid is selected, then the non-natural amino acids used to replace the plurality of initial amino acid residues may be identical to one another. For example, two histidines would be replaced by two non-natural amino acids having triazine-containing side chains. This approach will take advantage of the method for multisite incorporation of non-natural acids in proteins.

In another preferred embodiment, if more than one initial amino acid is selected, the non-natural amino acids used to replace the plurality of initial amino acid residues may be different from one another. For example, two histidines would be replaced by two different non-natural amino acids, e.g., one having a triazole group and the other with fluorinated triazole group. This approach may take advantage of the method for site-specific incorporation of non-natural amino acids using either stop codons or degenerate codons.

Conditional binding strategy is not just limited to the antigen-antibody interface. It can also be used to modify the Fc-region to have enhanced binding affinity to its receptors or to Clq of the complement system at the tumor site. These strategies will ensure that the downstream effector functions mediated by the antibody happen preferentially at the tumor sites rather than in healthy tissues, or while the antibody is in circulation. Similarly, the Fc interaction with its receptors can be designed to have a higher binding affinity at the lower pH. It has been reported that mutations causing weaker Fc/FcRn interaction result in a reduced Ab half life, while an increase in Fc/FcRn leads to increased serum half-life (see Martin et al., Molecular Cell 7: 867-877, 2001).

Also, tumor specific environment is not limited to the pH difference. Any of the other features of tumors can also be exploited to designing conditional binding.

In one embodiment, the antibody, when modified by the non-natural amino acids, has enhanced specificity and/or selectivity for the tumor tissue.

In one embodiment, the non-natural amino acid(s) comprises a side-chain that is not charged at pH of about 6.3-6.5.

In one embodiment, the natural amino acid(s) is histidine.

In one embodiment, the non-natural amino acid(s) is sterically similar to the natural amino acid(s).

In one embodiment, the non-natural amino acid(s) is sterically dissimilar to the natural amino acid(s).

In one embodiment, the method further comprises mutating amino acid(s) adjacent to the non-natural amino acid(s) for maintaining binding affinity and/or specificity of the protein.

In one embodiment, two or more natural amino acids in the protein are substituted with at least two different non-natural amino acids.

In one embodiment, two or more natural amino acids in the protein are substituted with the same non-natural amino acids.

In one embodiment, the non-natural amino acid(s) is incorporated into the protein by using a modified tRNA capable of being charged by both a natural amino acid and the non-natural amino acid.

In one embodiment, the non-natural amino acid(s) is incorporated into the protein in a site-specific manner by using a modified tRNA recognizing either stop codons or degenerate codons.

In one embodiment, the modified tRNA comprises a modified anticodon sequence that forms Watson-Crick base-pairing with a wobble degenerate codon for the natural amino acid.

In one embodiment, the modified tRNA is not charged substantially by an endogenous aminoacyl-tRNA synthetase (AARS) for the natural amino acid.

In one embodiment, the modified tRNA is charged by the endogenous AARS at a rate no more than 1% of that of its cognate tRNA.

In one embodiment, the modified tRNA is charged to carry the non-natural amino acid by a modified AARS with relaxed substrate specificity.

In one embodiment, the specificity constant (k_{cat}/K_{M}) for activation of the non-natural amino acid by the modified AARS is at least 5-fold larger than that for the natural amino acid.

In one embodiment, the modified tRNA further comprises a mutation at the fourth, extended anticodon site for increasing translation efficiency.

In one embodiment, the non-natural amino acid is incorporated into the protein at one or more specified position(s) by: (1) providing to a translation system a first polynucleotide encoding the subject modified tRNA; (2) providing to the translation system a second polynucleotide encoding a modified AARS with relaxed substrate specificity, or the modified AARS, wherein the modified AARS is capable of charging the modified tRNA with the non-natural amino acid; (3) providing to the translation system the non-natural amino acid; (4) providing to the translation system a template polynucleotide encoding the protein, wherein the codon(s) on the template polynucleotide for the specified position(s) forms Watson-Crick base-pairing with the modified tRNA; and, (5) allowing translation of the template polynucleotide, thereby incorporating the non-natural amino acid into the protein at the specified position(s), wherein steps (1)-(4) are effectuated in any order.

In one embodiment, the translation system is an in vitro translation system.

In one embodiment, the translation system is a cell.

In one embodiment, step (3) is effectuated by contacting the translation system with a solution containing the non-natural amino acid.

In one embodiment, the analog is provided by introducing additional nucleic acid construct(s) into the translation system, wherein the additional nucleic acid construct(s) encode one or more proteins required for biosynthesis of the non-natural amino acid.

In one embodiment, at least one of the additional nucleic acid construct(s) is operably linked to and subject to the control of an inducible promoter.

In one embodiment, the first and the second polynucleotides are encoded by a plasmid or plasmids.

In one embodiment, the first polynucleotide further comprises a first promoter sequence controlling the expression of the modified tRNA.

In one embodiment, the first promoter is an inducible promoter.

In one embodiment, the second polynucleotide further comprises a second promoter sequence controlling the expression of the modified AARS.

In one embodiment, the cell is auxotrophic for the natural amino acid encoded at the specified position.

In one embodiment, the translation system lacks endogenous tRNA that forms Watson-Crick base-pairing with the codon(s) at the specified position(s).

In one embodiment, the translation system is a cell, and the method further comprises disabling one or more genes encoding any endogenous tRNA that forms Watson-Crick base-pairing with the codon(s) at the specified position(s).

In one embodiment, the translation system is a cell, and the method further comprises inhibiting one or more endogenous AARS that charges tRNAs that form Watson-Crick base-pairing with the codon(s) at the specified position(s).

In one embodiment, the cell is a bacterial cell, an insect cell, a mammalian cell, or a fungal cell.

In one embodiment, the modified tRNA and/or the modified AARS are derived from a species different from that of the cell.

In one embodiment, the method further comprises verifying the incorporation of the non-natural amino acid.

In one embodiment, the incorporation of the non-natural amino acid is verified by mass spectrometry.

In one embodiment, the analog is incorporated into the position at an efficiency of at least about 50%.

In one embodiment, the non-natural amino acid(s) is selected from: 1,2,4-triazole-3-alanine, 2-fluoro-histidine, L-methyl histidine, 3-methyl-L-histidine, β-2-thienyl-L-alanine, or β-(2-Thiazolyl)-DL-alanine.

In one embodiment, the modified protein is a modified protein ligand, and wherein the binding partner is a cell-surface receptor, wherein the protein ligand undergoes receptor-mediated endocytosis.

In one embodiment, the modified protein binds the cell-surface receptor at a first pH, and does not substantially bind the cell-surface receptor at a second pH.

In one embodiment, the first and the second pH is at least about 0.5 pH unit apart, preferably about 1, 1.5, 2, 2.5, 3, 3.5, 4 or more pH units apart.

In one embodiment, the binding constant between the protein ligand and the cell-surface receptor at the first pH is at least about twice, three times, five times, 10 times, 20 times, 30 times, 50 times, 100 times, or 1000 times lower than that at the second pH.

In one embodiment, the first pH is the local extracellular pH of the protein ligand-cell surface receptor complex, and the second pH is endosomal pH.

In one embodiment, the protein ligand is a toxin or lectin selected from: Diptheria Toxin, Pseudomonas toxin, Cholera toxin, Ricin, or Concanavalin A; a viruses selected from: Rous sarcoma virus, Semliki forest virus, Vesicular stomatitis virus, or Adenovirus; a serum transport protein selected from: Transferrin, Low density lipoprotein, Transcobalamin, or Yolk protein; an antibody selected from: IgE, Polymeric IgA, Maternal IgG, or IgG (via Fc receptors); or a hormone or a growth factor selected from: insulin, EGF, Growth Hormone, Thyroid stimulating hormone, NGF, Calcitonin, Glucagon, Prolactin, Luteinizing Hormone, Thyroid hormone, PDGF, Interferon, or Catecholamine.

One aspect of the invention provides a method to modulate binding between a protein and a binding partner of the protein, the method comprising: introducing one or more non-natural amino acid(s) into the protein, wherein the non-natural amino acid(s) confers or substantially alters the pH-sensitive binding between the protein and the binding partner.

In one embodiment, the protein modified by the non-natural amino acid(s) becomes substantially able to bind the binding partner at a first pH, and becomes substantially unable to bind the binding partner at a second pH.

In one embodiment, the first and the second pH is at least about 0.5 pH unit apart, preferably about 1, 1.5, 2, 2.5, 3, 3.5, 4 or more pH units apart.

In one embodiment, the binding constant between the protein and the binding partner at the first pH is at least about twice, three times, five times, 10 times, 20 times, 30 times, 50 times, 100 times, or 1000 times lower than that at the second pH.

In one embodiment, the protein without the non-natural amino acid(s) becomes substantially able to bind the binding partner at a third pH, and becomes substantially unable to bind the binding partner at a fourth pH, and: (1) wherein the difference between the first and second pHs is at least about 0.5 units more or less than the difference between the third and fourth pHs, or (2) wherein the range between the first and second pH is shifted higher or lower to the same extent, and by at least about 0.5 pH units, compared to the range between the third and fourth pH.

In one embodiment, the first pH is the local extracellular pH of a pathological tissue, and the second pH is physiological pH.

In one embodiment, the first pH is about 6.3-6.5, and the second pH is about 7.6-7.8.

In one embodiment, the first pH is the local extracellular pH of a ligand-cell surface receptor complex, and the second pH is endosomal pH.

In one embodiment, the non-natural amino acid(s) is a histidine analog with a pH-sensitive side-chain.

In one embodiment, the histidine analog has a side-chain pKa at least 2-3 pH units lower than that of Histidine.

In one embodiment, the non-natural amino acid(s) is selected from: 1,2,4-triazole-3-alanine, 2-fluoro-histidine, L-methyl histidine, 3-methyl-L-histidine, β-2-thienyl-L-alanine, or β-(2-Thiazolyl)-DL-alanine.

In one embodiment, the non-natural amino acid(s) is incorporated into the binding interface between the protein and the binding partner.

In one embodiment, the non-natural amino acid(s) is incorporated into the protein in a site-specific manner.

In one embodiment, the non-natural amino acid(s) is incorporated into the protein using a degenerate codon orthogonal system.

A target protein of the antibody can be, for example, a tumor antigen, or an immune system effector molecule.

All embodiments described above are contemplated to be able to combine with one or more other embodiments, even for those described under different aspects of the invention.

### BRIEF DESCRIPTION OF THE FIGURES

FIG. 1 shows a schematic diagram for multiple-site-specific incorporation of non-natural amino acid into the UUU codon.

FIG. 2 shows the incorporation (or lack thereof) of NaI in place of Phe in several tryptic fragments of mDHFR, in response to the UUU codon. These data unambiguously establish that NaI incorporation is codon-biased to UUU.

FIG. 3 shows a schematic diagram for multiple-site-specific incorporation of non-natural amino acid into the UUG codon.

FIG. 4 demonstrates the replacement of Leu by NaI as detected in MALDI mass spectra of tryptic fragments of mDHFR.

FIG. 5 shows the effect of AZL on replacement of Leu by NaI as evaluated by MALDI mass spectra of tryptic fragments of mDHFR.

### DETAILED DESCRIPTION OF THE INVENTION

### I. Overview

In general, the instant invention provides methods and reagents for regulating protein interaction with its binding partner, which binding partner may be a protein itself, or other non-protein molecules (e.g. nucleic acid, lipid, polysaccharide, polymers, steroid, et.). More specifically, the invention provides methods and reagents for incorporating non-natural amino acids into a target protein, wherein the incorporated non-natural amino acids comprise side-chains that confer or substantially alter pH-sensitive binding to the binding partner by the target protein.

“Confer pH-sensitive binding,” as used herein, refers to the situation where the wild-type target protein binds to its binding partner in a relatively non-pH sensitive manner, at least within the pH ranges close to physiological pH conditions, such as about pH 4.5-9.5, preferably about pH 7.4-7.6; while the target protein modified by non-natural amino acids exhibits pH-sensitive binding to its binding partner, at least within the pH ranges close to physiological pH conditions, such as about pH 4.5-9.5. Obviously, pH-sensitive binding can occur over any range of pH values, include both physiological or non-physiological uses (such as non-biological use), so long as the relevant function of the molecule (e.g. enzyme, polymer, or other proteins) is not substantially impaired under these pH values.

“pH-sensitive binding” refers to the situation where binding affinity between two molecules (binding partners) change as environmental pH changes. For example, two molecules may bind each other at a first pH (or a first range of pH's), but exhibit progressively lower binding affinity as pH changes, till at one particular pH (e.g., the second pH), there is substantially reduced or even completely no binding between the two molecules. The reduction in binding affinity, as measured by binding constant Ka, may differ by at least about two, three, five, 10, 20, 30, 50, 100, 500, 1000 times or more. The first and second pH, under this scenario, may differ by at least about 0.5 pH unit, preferably about 1, 1.5, 2, 2.5, 3, 3.5, 4 or more pH units.

“Substantially alter pH-sensitive binding,” as used herein, refers to the situation where the non-natural amino acid(s), when incorporated into the target protein, substantially changes the pH-sensitive binding between the two binding partners. For example, if the un-modified protein becomes substantially able to bind its partner at a first pH, and becomes substantially unable to bind the same partner at a second pH, the difference between these two pH's may be a few pH units apart (e.g. 2 pH units). Upon incorporating the non-natural amino acids, the binding and non-binding pH may differ by more or less than the original 2 pH units. If this difference is at least about 0.5 pH unit, or at least about 1.0 unit, 1.5 units, 2.0 units or more, it can be said that there is a substantial alteration in pH-sensitive binding between the two binding partners. Alternatively, incorporation of non-natural amino acid(s) may shift (higher or lower) both the binding and the non-binding pH values, without changing the difference between the binding and the non-binding pH values. In this case, if the shift (in either direction) is at least about 0.5 pH unit, or at least about 1.0 unit, 1.5 units, 2.0 units or more, it can be said that there is a substantial alteration in pH-sensitive binding between the two binding partners.

While not wishing to be bound by any particular theory, pH-sensitive binding may partly result from the fact that certain amino acid side-chains may undergo change of net charged under different pH environments. That is, at a relatively high pH, certain side-chains may have no charge or only negative charge(s), while the same side-chains may possess positive or no charge, respectively, when the environmental pH becomes lower. This change of net charge may affect the interaction of a protein bearing such amino acid side-chains, since charged amino acid interaction is one of the most important forces that mediate protein-target interaction.

Again, while not wishing to be bound by any particular theory, the net charge change (from negative to less negative, neutral or even positive, from neutral to positive, from positive to more net positive charge, or vice versa, etc.) on the non-natural amino acid side chain under different pH values may bring about sufficient (some times even dramatic) structural changes of the molecule encompassing the non-natural amino acid, at least at/around the local environment of the non-natural amino acid, thus leading to changes in binding affinity/specificity/selectivity.

For example, if the binding partner of the protein interacts with the protein through a neutral surface that does not exhibit pH-sensitive binding, the binding surface (or elsewhere) of the protein may be engineered such that the binding interface is negatively charged at a higher (e.g. normal physiological) pH, but neutral at a relatively lower pH (e.g. target site). Presumably, the interaction between the protein and its binding partner is disfavored at normal pH due to the presence of the negative charge (in this case), and the interaction is favored at the target site because of the neutral-neutral interface. Alternatively, if the binding partner has a neutral binding surface at higher pH but positively-charged surface at lower pH, the protein may be designed to have a side-chain that is always negatively charged at both pH's. Many other scenarios can be envisioned based on analysis of particular binding interfaces on a case-by-case approach.

For example, Fc binds its receptor FcRn at slightly acidic pH of <6.5, allowing the transport of IgG to the blood stream. Once there, the environmental pH (about 7.4) loosens the binding, resulting in the release of IgG from the FcRn into the bloodstream (see Martin et al., Molecular Cell 7: 867-877, 2001). The mechanism of the pH-dependent FcRn/Fc affinity transition appears straightforward: FcRn binds to Fc with high affinity at pH<6.5 when Fc histidines 310, 435, and 436 are positively charged and binds to either Glu or Asp (all negatively charged) releases Fc upon deprotonation at pH values >7.0 (Glu and Asp on the binding partner FcRn still negatively charged). Martin et al. (supra). However, it is also found that transition of charges at different pH values on non-salt bridge Histidines may also contribute to the pH-sensitive binding between binding partners HFE and transferin receptor (see Martin et al., supra).

Ideally, to achieve preferred pH-sensitive binding between a normal physiological pH and a target site pH (either lower or higher than the physiological pH), the pKa of the amino acid side chain should be approximately in the middle between the physiological pH and the desired target pH.

Certain natural amino acids (such as Glu, Asp, His, Arg, Lys) have charged side-chains that may change net charge upon pH change. However, except for that of His, these side-chains have either extremely high or extremely low pKa's, and offers little choice or flexibility in terms of fine tuning pH-sensitive binding, at least within the physiological pH ranges. This is because the physiological pH is usually fixed around pH 7.6-7.8 (at least in human), only Histidine has a side-chain pH that is close to the physiological pH. However, certain pathological conditions (e.g. tumor) may create lower extracellular pH locally. Certain intracellular compartments (e.g. endosome) may inherently have lower pH. Unfortunately, the limited choice of charged natural amino acids, with their relatively extreme side-chain pKa's, makes it difficult to fine-tune the pH sensitive binding between a protein and its binding partner.

In contrast, a large number of non-natural amino acids have been successfully incorporated into proteins. These non-natural amino acids possess a diverse array of side-chains, which exhibit a broad range of side-chain pKa's that can be explored for different purposes. With this broad range of pKa's to choose from, it is likely that for any desirable target pH (higher or lower than the physiological pH), especially those that exist within a living organism, there will be one or more suitable non-natural amino acid side-chains that can be used to modulate pH-sensitive binding. Preferably, the side-chain pKa of the non-natural amino acid is between the normal and the target site pH, such as one close to the target site pH. In situations where it is desirable to promote binding at a target site, and inhibit binding at a third site (e.g. a particularly sensitive site with a different pH from the normal physiological pH), the side-chain pKa of the non-natural amino acid is preferably between the third and the target site pH.

Although there is no inherent limitation as to the position of the non-natural amino acids in the target protein, the most likely position for inserting or substituting the non-natural amino acids is likely the binding interface, where the non-natural amino acid may directly participate in interacting with moieties on the binding partner.

In one embodiment, the target protein is a tumor-targeting antibody including one or more non-natural amino acid(s).

In certain embodiments, the non-natural amino acids confer the modified antibodies with enhanced specificity/affinity/selectivity for its binding targets in low pH environments (such as neoplastic tissue, bone marrow, etc.), hypoxia conditions, etc.

For those embodiments relating to enhanced specificity/affinity/selectivity for targets in low pH environments, the invention is partially based on the discovery that certain tissues in pathological conditions, such as neoplastic or tumor tissues in vivo, present a relatively lower extracellular pH (e.g., at least about 0.5 pH unit lower, typically about 1-1.5 pH units lower) than that of normal tissues under physiological conditions (e.g. about pH 7.6-7.8). Thus modified antibodies with non-natural amino acids, which possess unique side-chains, may preferentially bind to tissues in such low pH environments (such as the tumor tissues). This is beneficial since many antibodies, including those FDA-approved or other commercially marketed antibodies are not strictly tumor-specific, despite the fact that tumor tissues may over-express targets for these antibodies. One example of such, the HER-2/neu monoclonal antibody HERCEPTIN, is shown below in the examples section. Numerous other commercially available or FDA-approved monoclonal antibodies may be improved using the subject method.

Thus the modified antibody of the invention has enhanced tumor-specificity/selectivity due to a mechanism independent of antigen specificity per se.

Similarly, non-natural amino acids with other unique side-chain properties may be incorporated into antibodies to confer tissue-specificity based on other mechanisms, such as hypoxia at tumor sites, and the presence of tumor-specific extracellular enzymes. For example, due to the collectively diverse but individually unique side-chain chemistry, non-natural amino acids may be selected for incorporation into antibodies to confer selectivity/specificity/affinity for hypoxia tissues. Such non-natural amino acid side-chains may enhance binding of the antibody to its antigen under a reducing environment.

The non-natural amino acids may be incorporated anywhere within the antibody, but preferably incorporated in the antigen-binding region of the monoclonal antibody, such that the modified antibody may have a higher binding affinity for its targets in the tumor environment than in non-tumor environments.

The non-natural amino acids may be inserted at a desired location. This can be achieved by inserting a codon for the non-natural amino acid (see below) in the polynucleotide encoding the antibody. Alternatively, the non-natural amino acids may be incorporated by substitution of a natural amino acid using, for example, one of the methods described below or any other methods known in the art.

In certain embodiments, a single non-natural amino acid is incorporated per antibody.

In certain embodiments, two or more identical or different non-natural amino acids may be incorporated per antibody.

In certain embodiments, the non-natural amino acids are incorporated in a site-specific manner. For example, the non-natural amino acids may substitute specific natural amino acids (e.g. His) at specific locations, even though the same kind of natural amino acid at other locations of the antibody are not substituted.

In certain embodiments, the non-natural amino acids are incorporated in a non-site-specific manner. For example, all His residues of the antibody may be non-specifically replaced by one or more non-natural amino acids.

If the non-natural amino acid is incorporated by substituting natural amino acids, any of the 20 natural amino acids may be replaced. In a preferred embodiment, His residues may be replaced by non-natural amino acids.

The structure of the non-natural amino acids and the replaced natural amino acids may be similar or dissimilar. For example, His residues may be replaced by a non-natural His analog. Alternatively, Ala (or for that matter, any other 19 amino acids) may be replaced by a non-natural His analog.

Numerous non-natural amino acids have been successfully incorporated into proteins. Any of these non-natural amino acids or their analogs may be tested for their side-chain pKa's. Non-natural amino acids with desirable side-chain properties, such as stronger affinity for low-pH environment, may be selected for incorporation into the subject antibodies.

Thus non-natural amino acids might be obtained by modifying the structure of a natural amino acid, or another non-natural amino acid. Alternatively, the non-natural amino acids may be obtained by screening a library of non-natural amino acids for those with desired side-chain pKa values (for example, a lower pKa of about 2.5-4.5).

Since the pKa of any amino acid side-chain may be different when it is incorporated into a protein, selection of non-natural amino acids may not be limited to measuring the pKa of a non-natural amino acid monomer, but may also include a further verification step of measuring the pKa of the non-natural amino acid when it is incorporated into the antibody.

Non-natural amino acids may be incorporated into protein using various methods. For example, in one embodiment, if the non-natural amino acid is structurally/sterically similar to one of the twenty natural amino acids, the non-natural amino acid may be incorporated into a target protein by way of competitive biosynthetic assimilation (See Budisa 1995, Eur. J. Biochem 230: 788-796; Deming 1997, J. Macromol. Sci. Pure Appl. Chem. A34; 2143-2150; Duewel 1997, Biochemistry 36: 3404-3416; van Hest and Tirrell 1998, FEBS Lett 428(1-2): 68-70; Sharma et al., 2000, FEBS Lett 467(1): 37-40. All incorporated herein by reference).

In certain embodiments, the competing natural amino acids might be selectively depleted to enhance the incorporation of non-natural amino acids.

In another embodiment, non-natural amino acids may be incorporated into antibodies by using either a nonsense suppressor or a frame-shift suppressor tRNA in response to amber or four-base codons, respectively (See Bain et al., J. Am. Chem. Soc. 111: 8013, 1989; Noren et al., Science 244: 182, 1989; Furter, Protein Sci. 7: 419, 1998; Wang et al., Proc. Natl. Acad. Sci. U.S.A., 100: 56, 2003; Hohsaka et al., FEBS Lett. 344: 171: 1994; Kowal and Oliver, Nucleic Acids Res. 25: 4685, 1997. All incorporated herein by reference). Such methods insert non-canonical amino acids at codon positions that will normally terminate wild-type peptide synthesis (e.g. a stop codon or a frame-shift mutation). These methods have worked well for single-site insertion of novel amino acids. These methods may work modestly well for multisite incorporation, if modest (20-60%) suppression efficiencies are acceptable (See Anderson et al., J. Am. Chem. Soc. 124: 9674, 2002; Bain et al., Nature 356: 537, 1992; Hohsaka et al., Nucleic Acids Res. 29: 3646, 2001. All incorporated herein by reference).

In yet another embodiment, efficient multisite incorporation may be accomplished by replacement of natural amino acids in auxotrophic Escherichia coli strains, and by using aminoacyl-tRNA synthetases with relaxed substrate specificity or attenuated editing activity (See Wilson and Hatfield, Biochim. Biophys. Acta 781: 205, 1984; Kast and Hennecke, J. Mol. Biol. 222: 99, 1991; Ibba et al., Biochemistry 33: 7107, 1994; Sharma et al., FEBS Lett. 467: 37, 2000; Tang and Tirrell, Biochemistry 41: 10635, 2002; Datta et al., J. Am. Chem. Soc. 124: 5652, 2002; Doring et al., Science 292: 501, 2001. All incorporated herein by reference). This method may be useful, particularly when it is acceptable to allow non-natural amino acids to “share” codons with one of the natural amino acids, and when incorporation at an unintended site does not substantially compromise the function of the antibody.

In a preferred embodiment, the non-natural amino acids may be incorporated into in s site-specific manner into an antibody, by utilizing a system that breaks the genetic codon degeneracy (see details below).

The sections below describe in further details certain aspects of the invention. All examples are for illustrative purpose only, and not intended to be limiting in any respect.

### Non-Natural Amino Acid-Containing Protein Expression

As described above, the subject non-natural amino acid residues can be incorporated into proteins in a variety of ways. This section provides illustrative details for a few such methods.

In one of the embodiments, for evaluative purposes, an antibody fragment containing non-natural amino acids can be directly synthesized chemically using solid phase synthesis and ligation technologies, or using in vitro translation/expression. The intact antibody or its fragments can also be expressed using a variety of well-established protein expression systems including E. coli, yeasts, insect (e.g. baculo-virus system), and mammalian cells.

In a preferred embodiment, for site-specific multisite incorporation of non-natural amino acids, a procedure described in patent application publication US 20020042097 (entire content incorporated herein by reference) may be used.

Briefly, US 20020042097 provides a general method for producing a modified polypeptide (e.g. the subject Antibody or functional fragments, derivatives thereof), wherein the polypeptide is modified by replacing a selected amino acid with a desired amino acid analogue (e.g. non-natural amino acid), which method comprises:

(a) transforming a host cell with: i) a vector having a polynucleotide sequence encoding an aminoacyl-tRNA synthetase for the selected/natural amino acid; and ii) a vector having a polynucleotide sequence encoding a polypeptide molecule of interest (e.g. the subject Antibody or functional fragments, derivatives thereof) so as to produce a host vector system; wherein the vectors of (i) and (ii) may be the same or different;

(b) growing the host-vector system in a medium which comprises the selected amino acid, so that the host vector system overexpresses the aminoacyl-tRNA synthetase;

(c) replacing the medium with a medium which lacks the selected amino acid and has the desired amino acid analogue;

(d) growing the host vector system in the medium which lacks the selected amino acid and has the desired amino acid analogue under conditions so that the host vector system overexpresses the polypeptide molecule of interest and the selected amino acid is replaced with the desired amino acid analogue, thereby producing the modified polypeptide.

According to this method, overexpression of an aminoacyl-tRNA synthetase results in an increase in the activity of the aminoacyl-tRNA synthetase. This method is partially based on the discovery that incorporation of non-natural amino acid analogues into polypeptides can be improved in cells that overexpress aminoacyl-tRNA synthetases (AARSs) that recognize such amino acid analogues as substrates. “Improvement” is defined as either increasing the scope of amino acid analogues (i.e. kinds of amino acid analogues) that can be incorporated, or by increasing the yield of the modified polypeptide. Overexpression of the aminoacyl-tRNA synthetase increases the level of aminoacyl-tRNA synthetase activity in the cell. The increased activity leads to an increased rate of incorporation of non-natural amino acid analogues into the growing peptide, thus the increased rate of synthesis of the polypeptides, thereby increasing the quantity of polypeptides containing such non-natural amino acid analogues, i.e. modified polypeptides, produced by the subject method.

The nucleic acids, encoding the aminoacyl-tRNA synthetase, and the nucleic acids encoding the polypeptide of interest (antibody or its fragment), may be located in the same or different vectors. The vectors include expression control elements which direct the production of the aminoacyl-tRNA synthetase, and the polypeptide of interest. The expression control elements (i.e. regulatory sequences) can include inducible promoters, constitutive promoters, secretion signals, enhancers, transcription terminators, and other transcriptional regulatory elements.

In the host-vector system, the production of an aminoacyl-tRNA synthetase (histidyl tRNA synthetase) can be controlled by a vector which comprises expression control elements that direct the production of the aminoacyl-tRNA synthetase. Preferably, the production of aminoacyl-tRNA synthetase is in an amount in excess of the level of naturally occurring aminoacyl-tRNA synthetase, such that the activity of the aminoacyl-tRNA synthetase is greater than naturally occurring levels.

In the host-vector system, the production of an aminoacyl-tRNA synthetase (histidyl tRNA synthetase) can be controlled by a vector which comprises expression control elements that direct the production of the aminoacyl-tRNA synthetase. Preferably, the production of aminoacyl-tRNA synthetase is in an amount in excess of the level of naturally occurring aminoacyl-tRNA synthetase, such that the activity of the aminoacyl-tRNA synthetase is greater than naturally occurring levels.

In the host-vector system, the production of a antibody or its fragment can be controlled by a vector which comprises expression control elements for producing the antibody or its fragment of interest. Preferably, the polypeptide of interest (e.g. Ab) so produced is in an amount in excess of the level produced by a naturally occurring gene encoding the polypeptide of interest.

The host-vector system can be constitutively overexpressing the aminoacyl-tRNA synthetase and induced to overexpress the polypeptide of interest (e.g. Ab) by contacting the host-vector system with an inducer, such as isopropyl-beta-D-thiogalactopyranoside (IPTG). The host-vector system can also be induced to overexpress the aminoacyl-tRNA synthetase and/or the protein of interest by contacting the host-vector system with an inducer, such as IPTG. Other inducers include stimulation by an external stimulation such as heat shock.

Using the methods of the invention, any natural amino acid can be selected for replacement by a non-natural amino acid analogue in the polypeptide of interest. A non-natural amino acid analogue is preferably an analogue of the natural amino acid to be replaced. To replace a selected natural amino acid with an amino acid analogue in a polypeptide of interest, an appropriate corresponding aminoacyl tRNA synthetase must be selected. For example, if an amino acid analogue will replace a methionine residue, then preferably a methionyl tRNA synthetase is selected.

In one embodiment, the host-vector system is grown in media lacking the natural amino acid and supplemented with a non-natural amino acid analogue, thereby producing a modified polypeptide (e.g. Ab) that has incorporated at least one non-natural amino acid analogue. This method is superior to existing methods as it improves the efficiency of incorporating amino acid analogues into polypeptides of interest, and it increases the quantity of the modified polypeptides so produced.

Various host cells may be used for this method, including those of bacterial, yeast, mammalian, insect, or plant cells. Preferably, the host cell is an auxotroph (such as a methionine auxotroph), which is incapable of producing the selected amino acid.

According to this embodiment, the host-vector system is initially grown in media which includes all essential amino acids, induced to express the polypeptide of interest, and subsequently after induction, is grown in media lacking the natural amino acid and supplemented with a non-natural amino acid analogue, thereby producing a modified polypeptide that has incorporated at least one non-natural amino acid analogue.

For example, the method of the invention can be practiced by: (1) growing the host-vector system under suitable conditions having the natural amino acid and under conditions such that the host vector system overexpresses the aminoacyl-tRNA synthetase; (2) collecting and washing cells to remove presence of the natural amino acid; (3) resuspending the cells in media medium which lacks the natural amino acid and has an amino acid analogue; (4) inducing the expression of the polypeptide of interest; (5) growing the cells in a medium which lacks the natural amino acid and has an amino acid analogue under conditions such that the host-vector system overexpresses the aminoacyl-tRNA synthetase and the polypeptide molecule of interest; and (6) isolating the modified polypeptide of interest.

The aminoacyl-tRNA synthetase (such as methionyl tRNA synthetase) may be naturally occurring or genetically engineered.

Certain of the selected natural amino acids may have their codon mutated to eliminate incorporation of non-natural amino acids at such codons, to achieve at least partial site-specificity of non-natural amino acid incorporation.

In yet another preferred embodiment, for site specific incorporation of non-natural amino acids, a degenerate codon orthogonal system can be used. The methods for the procedure are described in Breaking the degeneracy of the genetic code. Kwon I, Kirshenbaum K, Tirrell D A, J Am Chem. Soc. 2003 Jun. 25; 125(25):7512-3 (incorporated herein by reference). This method incorporates the subject non-natural amino acids into the subject antibodies in a site-specific manner by using an expression system comprising modified AARS that can charge the desired non-natural amino acids onto a modified tRNA with altered anti-codon loop.

The methods and compositions provide a means for site-specific incorporation of non-natural amino acids directly into proteins in vivo. Importantly, the non-natural amino acid is added to the genetic repertoire, rather than substituting for one of the common 20 amino acids.

The general method, e.g., (i) allows the site-selective insertion of one or more non-natural amino acids at any desired position of any protein, (ii) is applicable to both prokaryotic and eukaryotic cells, (iii) enables in vivo studies of mutant proteins in addition to the generation of large quantities of purified mutant proteins, and (iv) is adaptable to incorporate any of a large variety of non-natural amino acids, into proteins in vivo. Thus, in a specific polypeptide sequence a number of different site-selective insertions of non-natural amino acids is possible. Such insertions are optionally all of the same type (e.g., multiple examples of one type of non-natural amino acid inserted at multiple points in a polypeptide) or are optionally of diverse types (e.g., different non-natural amino acid types are inserted at multiple points in a polypeptide).

The method partly depend on the use of a modified tRNA based on a wild-type tRNA for a natural amino acid.

In certain embodiments, the natural amino acid is encoded by two or more genetic codes (thus encoded by degenerate genetic codes). In most, if not all cases, this includes 18 of the 20 natural amino acids, except Met and Trp. In these circumstances, to recognize all the degenerate genetic codes for the natural amino acid, the anticodon loop of the wild-type tRNA(s) relies on both wobble base-pairing and pure Watson-Crick base-pairing. The subject modified tRNA contains at least one modification in its anticodon loop, such that the modified anticodon loop now forms Watson-Crick base-pairing to one of the degenerate genetic codes, which the tRNA previously bind only through wobble base-pairing (see Example I below).

Since Watson-Crick base pairing is invariably stronger and more stable than wobble base pairing, the subject modified tRNA will preferentially bind to a previous wobble base-pairing genetic code (now through Watson-Crick base-pairing), over a previous Watson-Crick base-pairing (now through wobble base-pairing). Thus an analog may be incorporated at the subject codon, if the modified tRNA is charged with an analog of a natural amino acid, which may or may not be the same as the natural amino acid encoded by the codon in question.

For example, in Example II below, some Phe in mouse DHFR (mDHFR) are encoded by UUC codons, some others by UUU codons. The wild-type E. coli tRNA for Phe has a GAA anticodon sequence, and thus binds the UUC codons through Watson-Crick base-pairing, and binds the UUU codons through wobble base-pairing. Thus in E. coli, a modified tRNA, such as a yeast tRNA for Phe may have a modified anticodon sequence of AAA, so that it now preferentially binds to the previously “disfavored” UUU codons. When such a modified Phe tRNA is charged with NaI, it competes with the wild-type Phe tRNA charged with Phe for the UUU codon. But since the modified tRNA binds UUU through the stronger Watson-Crick base-pairing, NaI (rather than Phe) will be preferentially, if not exclusively, inserted in the UUU codons.

In fact, the anticodon sequence of the modified tRNA may be changed in such a way that it now recognizes a codon for a different natural amino acid. For example, in Example III, the Phe tRNA anticodon sequence is changed from GAA to CAA, which is capable of Watson-Crick base-pairing with a Leu (rather than a Phe) codon UUG. Such a modified Phe tRNA can now incorporate NaI into certain Leu codons.

Thus in certain embodiments, if it is desirable to incorporate certain amino acid analogs at codons for Met or Trp, a tRNA for a natural amino acid (e.g., a Met tRNA, a Trp tRNA, or even a Phe tRNA, etc.) may be modified to recognize the Met or Trp codon. Under this type of unique situation, both the modified tRNA and the natural tRNA compete to bind the same (single) genetic code through Watson-Crick base-pairing. Some but not all such codons will accept their natural amino acids, while others may accept amino acid analogs carried by the modified tRNA. Other factors, such as the abundance of the natural amino acid vs. that of the analog, may affect the final outcome.

This also applies to other situations where a modified tRNA competes with wild-type tRNA for any natural amino acids. Such modified tRNAs are within the scope of the instant invention.

In certain preferred embodiments, the modified tRNA is not charged or only inefficiently charged by an endogenous aminoacyl-tRNA synthetase (AARS) for any natural amino acid, such that the modified tRNA largely (if not exclusively) carries an amino acid analog, but not a natural amino acid. Although a subject modified tRNA may still be useful if it can be charged by the endogenous AARS with a natural amino acid.

In certain embodiments, the modified tRNA charged with an amino acid analog has such an overall shape and size that the analog-tRNA is a ribosomally acceptable complex, that is, the tRNA-analog complex can be accepted by the prokaryotic or eukaryotic ribosomes in an in vivo or in vitro translation system.

In certain embodiments, the modified tRNA can be efficiently charged to carry an analog of a natural amino acid. The amino acid analog may be a derivative of at least one of the 20 natural amino acids, with one or more functional groups not present in natural amino acids. For example, the functional group may be selected from the group consisting of: bromo-, iodo-, ethynyl-, cyano-, azido-, aceytyl, aryl ketone, a photo labile group, a fluoresent group, and a heavy metal, so long as the side chain property (such as pKa) renders the non-natural amino acid suitable to confer or substantially alter pH-sensitive binding.

In certain embodiments, the modified tRNA can be charged to carry the analog by a modified AARS with relaxed substrate specificity.

Preferably, the modified AARS specifically or preferentially charges the non-natural amino acid/analog to the modified tRNA over any natural amino acid. In a preferred embodiment, the specificity constant for activation of the analog by the modified AARS (defined as k_{cat}/K_{M}) is at least about 2-fold larger than that for the natural amino acid, preferably about 3-fold, 4-fold, 5-fold or more than that for the natural amino acid.

In certain embodiments, the modified tRNA further comprises a mutation at the fourth, extended anticodon site for increase translational efficiency.

In certain embodiments, the modified tRNA is charged by the endogenous AARS at a rate no more than about 50%, 30%, 20%, 10%, 5%, 2%, or 1% of that of the tRNA.

Another aspect of the invention provides a modified tRNA encoded by any one of the subject polynucleotides.

Another aspect of the invention provides a method for incorporating the subject non-natural amino acid analog into a target protein (e.g. the subject Ab or its functional derivatives, fragments, fusion proteins, etc.) at one or more specified positions, the method comprising: (1) providing to an environment a first polynucleotide encoding a modified tRNA, or a subject modified tRNA; (2) providing to the environment a second polynucleotide encoding a modified AARS with relaxed substrate specificity, or the modified AARS, wherein the modified AARS is capable of charging the modified tRNA with the desired non-natural amino acid/analog; (3) providing to the environment the analog; (4) providing a template polynucleotide encoding the target protein, wherein the codon on the template polynucleotide for the specified position only forms Watson-Crick base-pairing with the modified tRNA; and, (5) allowing translation of the template polynucleotide to proceed, thereby incorporating the analog into the target protein at the specified position, wherein steps (1)-(4) are effectuated in any order.

In certain embodiments, the methods of the invention involve introducing into an environment (e.g., a cell or an in vitro translation system (IVT)) a first nucleic acid encoding an orthogonal/modified tRNA molecule that is not charged efficiently by an endogenous aminoacyl-tRNA synthetase in the cell/in vitro translation system (IVT), or the orthogonal/modified tRNA itself. The orthogonal/modified tRNA molecule has an anticodon complementary to a degenerate codon sequence, which is one of a plurality of codon sequences encoding a naturally occurring amino acid. Such a codon is said to be degenerate. According to the methods of this embodiment of the invention, a second nucleic acid encoding an orthogonal/modified aminoacyl tRNA synthetase (AARS) is also introduced into the cell/IVT. The orthogonal/modified AARS is capable of charging the orthogonal/modified tRNA with a chosen amino acid analog. The amino acid analog can then be provided to the cell so that it can be incorporated into one or more proteins within the cell or IVT.

Thus in certain embodiments, the environment is an in vitro translation system. For example, suitable IVT systems include the Wheat Germ Lysate-based PROTEINscript-PRO™, Ambion's E. coli system for coupled in vitro transcription/translation; or the rabbit reticulocyte lysate-based Retic Lysate IVT™ Kit from Ambion). Optionally, the in vitro translation system can be selectively depleted of one or more natural AARSs (by, for example, immunodepletion using immobilized antibodies against natural AARS) and/or natural amino acids so that enhanced incorporation of the analog can be achieved. Alternatively, nucleic acids encoding the re-designed AARSs may be supplied in place of recombinantly produced AARSs. The in vitro translation system is also supplied with the analogs to be incorporated into mature protein products.

In other embodiments, the environment is a cell. A variety of cells (or lysates thereof suitable for IVT) can be used in the methods of the invention, including, for example, a bacterial cell, a fungal cell, an insect cell, and a mammalian cell (e.g. a human cell or a non-human mammal cell). In one embodiment, the cell is an E. coli cell.

In certain embodiments, the amino acid analog can be provided by directly contacting the cell or IVT with the analog, for example, by applying a solution of the analog to the cell in culture, or by directly adding the analog to the IVT. The analog can also be provided by introducing one or more additional nucleic acid construct(s) into the cell/IVT, wherein the additional nucleic acid construct(s) encodes one or more amino acid analog synthesis proteins that are necessary for synthesis of the desired analog.

In certain embodiments, the additional nucleic acid construct(s) has an inducible promoter sequence that can induce expression of the one or more synthesis proteins.

The methods of this embodiment of the invention further involve introducing a template nucleic acid construct into the cell/IVT, the template encoding a protein, wherein the nucleic acid construct contains at least one degenerate codon sequence.

The nucleic acids introduced into the cell/IVT can be introduced as one construct or as a plurality of constructs. In certain embodiments, the various nucleic acids are included in the same construct. For example, the nucleic acids can be introduced in any suitable vectors capable of expressing the encoded tRNA and/or proteins in the cell/IVT. In one embodiment, the first and second nucleic acid sequences are provided in one or more plasmids. In another embodiment, the vector or vectors used are viral vectors, including, for example, adenoviral and lentiviral vectors. The sequences can be introduced with an appropriate promoter sequence for the cell/IVT, or multiple sequences that can be inducible for controlling the expression of the sequences.

In certain embodiments, the plasmid or plasmids containing the subject polynucleotides have one or more selectable markers, such as antibiotic resistance genes.

In certain embodiments, the first polynucleotide further comprises a first promoter sequence controlling the expression of the modified tRNA. The first promoter is an inducible promoter.

In certain embodiments, the second polynucleotide further comprises a second promoter sequence controlling the expression of the modified AARS.

In certain embodiments, the cell is auxotrophic for the amino acid naturally encoded by the degenerate codon.

In certain embodiments, the cell is auxotrophic for the natural amino acid encoded at the specified position.

In certain embodiments, the environment lacks endogenous tRNA that forms Watson-Crick base-pairing with the codon at said specified position.

When the cell has a tRNA that has an anticodon perfectly complementary to the degenerate codon, the methods can include a step of disabling the gene encoding such an endogenous tRNA.

Alternatively, the environment is a cell, and the method further comprises inhibiting one or more endogenous AARS that charges tRNAs that form Watson-Crick base-pairing with the codon.

In certain embodiments, the orthogonal tRNA and orthogonal aminoacyl tRNA-synthetase can be derived from an organism from a different species than that of the cell/the IVT. For example, a yeast tRNA and a yeast AARS may be used with an E. coli cell.

In certain embodiments, the method further comprises verifying the incorporation of the analog by, for example, mass spectrometry.

In certain embodiments, the method incorporates the analog into the position at an efficiency of at least about 50%, or 60%, 70%, 80%, 90%, 95%, 99% or nearly 100%.

Other embodiments or aspects of the invention are further described in the sections below.

### II. Definitions

Aspects and embodiments of the instant invention is not limited to particular compositions or biological systems, which can, of course, vary. It is also to be understood that the terminology used herein is for the purpose of describing particular illustrative embodiments only, and is not intended to be limiting. As used in this specification and the appended claims, the singular forms “a,” “an,” and “the” include plural referents unless the content clearly dictates otherwise. Thus, for example, reference to “a molecule” optionally includes a combination of two or more such molecules, and the like.

Unless specifically defined below, the terms used in this specification generally have their ordinary meanings in the art, within the general context of this invention and in the specific context where each term is used. Certain terms are discussed below or elsewhere in the specification, to provide additional guidance to the practitioner in describing the compositions and methods of the invention and how to make and use them. The scope an meaning of any use of a term will be apparent from the specific context in which the term is used.

“About” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“Amino acid analog,” “non-canonical amino acid,” or “non-standard amino acid,” used interchangeably, is meant to include all amino acid-like compounds that are similar in structure and/or overall shape to one or more of the twenty L-amino acids commonly found in naturally occurring proteins (Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y, as defined and listed in WIPO Standard ST.25 (1998), Appendix 2, Table 3, incorporated herein by reference). Amino acid analog can also be natural amino acids with modified side chains or backbones. Preferably, these analogs usually are not “substrates” for the amino acyl tRNA synthethases (AARSs) because of the normally high specificity of the AARSs. Although occasionally, certain analogs with structures/shapes sufficiently close to those of natural amino acids may be erroneously incorporated into proteins by AARSs, especially modified AARSs with relaxed substrate specificity. In a preferred embodiment, the analogs share backbone structures, and/or even the most side chain structures of one or more natural amino acids, with the only difference(s) being containing one or more modified groups in the molecule. Such modification may include, without limitation, substitution of an atom (such as N) for a related atom (such as S), addition of a group (such as methyl, or hydroxyl group, etc.) or an atom (such as Cl or Br, etc.), deletion of a group (supra), substitution of a covalent bond (single bond for double bond, etc.), or combinations thereof. Amino acid analogs may include α-hydroxy acids, and β-amino acids, and can also be referred to as “modified amino acids,” or “non-natural AARS substrates.”

The amino acid analogs may either be naturally occurring or non-naturally occurring (e.g. synthesized). As will be appreciated by those in the art, any structure for which a set of rotamers is known or can be generated can be used as an amino acid analog. The side chains may be in either the (R) or the (S) configuration (or D- or L-configuration). In a preferred embodiment, the amino acids are in the (S) or L-configuration.

Preferably, the overall shape and size of the amino acid analogs are such that, upon being charged to (natural or re-designed) tRNAs by (natural or re-designed) AARS, the analog-tRNA is a ribosomally accepted complex, i.e., the tRNA-analog complex can be accepted by the prokaryotic or eukaryotic ribosomes in an in vivo or in vitro translation system.

“Backbone,” or “template” includes the backbone atoms and any fixed side chains (such as the anchor residue side chains) of the protein (e.g., AARS). For calculation purposes, the backbone of an analog is treated as part of the AARS backbone.

“Protein backbone structure” or grammatical equivalents herein is meant the three dimensional coordinates that define the three dimensional structure of a particular protein. The structures which comprise a protein backbone structure (of a naturally occurring protein) are the nitrogen, the carbonyl carbon, the α-carbon, and the carbonyl oxygen, along with the direction of the vector from the α-carbon to the β-carbon.

The protein backbone structure which is input into the computer can either include the coordinates for both the backbone and the amino acid side chains, or just the backbone, i.e. with the coordinates for the amino acid side chains removed. If the former is done, the side chain atoms of each amino acid of the protein structure may be “stripped” or removed from the structure of a protein, as is known in the art, leaving only the coordinates for the “backbone” atoms (the nitrogen, carbonyl carbon and oxygen, and the α-carbon, and the hydrogens attached to the nitrogen and α-carbon).

Optionally, the protein backbone structure may be altered prior to the analysis outlined below. In this embodiment, the representation of the starting protein backbone structure is reduced to a description of the spatial arrangement of its secondary structural elements. The relative positions of the secondary structural elements are defined by a set of parameters called supersecondary structure parameters. These parameters are assigned values that can be systematically or randomly varied to alter the arrangement of the secondary structure elements to introduce explicit backbone flexibility. The atomic coordinates of the backbone are then changed to reflect the altered supersecondary structural parameters, and these new coordinates are input into the system for use in the subsequent protein design automation. For details, see U.S. Pat. No. 6,269,312, the entire content incorporated herein by reference.

“Conformational energy” refers generally to the energy associated with a particular “conformation”, or three-dimensional structure, of a macromolecule, such as the energy associated with the conformation of a particular protein. Interactions that tend to stabilize a protein have energies that are represented as negative energy values, whereas interactions that destabilize a protein have positive energy values. Thus, the conformational energy for any stable protein is quantitatively represented by a negative conformational energy value. Generally, the conformational energy for a particular protein will be related to that protein's stability. In particular, molecules that have a lower (i.e., more negative) conformational energy are typically more stable, e.g., at higher temperatures (i.e., they have greater “thermal stability”). Accordingly, the conformational energy of a protein may also be referred to as the “stabilization energy.”

Typically, the conformational energy is calculated using an energy “force-field” that calculates or estimates the energy contribution from various interactions which depend upon the conformation of a molecule. The force-field is comprised of terms that include the conformational energy of the alpha-carbon backbone, side chain-backbone interactions, and side chain-side chain interactions. Typically, interactions with the backbone or side chain include terms for bond rotation, bond torsion, and bond length. The backbone-side chain and side chain-side chain interactions include van der Waals interactions, hydrogen-bonding, electrostatics and solvation terms. Electrostatic interactions may include coulombic interactions, dipole interactions and quadrapole interactions). Other similar terms may also be included. Force-fields that may be used to determine the conformational energy for a polymer are well known in the art and include the CHARMM (see, Brooks et al, J. Comp. Chem. 1983, 4:187-217; MacKerell et al., in The Encyclopedia of Computational Chemistry, Vol. 1:271-277, John Wiley & Sons, Chichester, 1998), AMBER (see, Cornell et al., J. Amer. Chem. Soc. 1995, 117:5179; Woods et al., J. Phys. Chem. 1995, 99:3832-3846; Weiner et al., J. Comp. Chem. 1986, 7:230; and Weiner et al., J. Amer. Chem. Soc. 1984, 106:765) and DREIDING (Mayo et al., J. Phys. Chem. 1990, 94-:8897) force-fields, to name but a few.

In a preferred implementation, the hydrogen bonding and electrostatics terms are as described in Dahiyat & Mayo, Science 1997 278:82). The force field can also be described to include atomic conformational terms (bond angles, bond lengths, torsions), as in other references. See e.g., Nielsen J E, Andersen K V, Honig B, Hooft R W W, Klebe G, Vriend G, & Wade R C, “Improving macromolecular electrostatics calculations,” Protein Engineering, 12: 657662(1999); Stikoff D, Lockhart D J, Sharp K A & Honig B, “Calculation of electrostatic effects at the amino-terminus of an alpha-helix,” Biophys. J., 67: 2251-2260 (1994); Hendscb Z S, Tidor B, “Do salt bridges stabilize proteins—a continuum electrostatic analysis,” Protein Science, 3: 211-226 (1994); Schneider J P, Lear J D, DeGrado W F, “A designed buried salt bridge in a heterodimeric coil,” J. Am. Chem. Soc., 119: 5742-5743 (1997); Sidelar C V, Hendsch Z S, Tidor B, “Effects of salt bridges on protein structure and design,” Protein Science, 7: 1898-1914 (1998). Solvation terms could also be included. See e.g., Jackson S E, Moracci M, elMastry N, Johnson C M, Fersht A R, “Effect of Cavity-Creating Mutations in the Hydrophobic Core of Chymotrypsin Inhibitor 2,” Biochemistry, 32: 11259-11269 (1993); Eisenberg, D & McLachlan A D, “Solvation Energy in Protein Folding and Binding,” Nature, 319: 199-203 (1986); Street A G & Mayo S L, “Pairwise Calculation of Protein Solvent-Accessible Surface Areas,” Folding & Design, 3: 253-258 (1998); Eisenberg D & Wesson L, “Atomic solvation parameters applied to molecular dynamics of proteins in solution,” Protein Science, 1: 227-235 (1992); Gordon & Mayo, supra.

“Coupled residues” are residues in a molecule that interact, through any mechanism. The interaction between the two residues is therefore referred to as a “coupling interaction.” Coupled residues generally contribute to polymer fitness through the coupling interaction. Typically, the coupling interaction is a physical or chemical interaction, such as an electrostatic interaction, a van der Waals interaction, a hydrogen bonding interaction, or a combination thereof. As a result of the coupling interaction, changing the identity of either residue will affect the “fitness” of the molecule, particularly if the change disrupts the coupling interaction between the two residues. Coupling interaction may also be described by a distance parameter between residues in a molecule. If the residues are within a certain cutoff distance, they are considered interacting.

“Expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

“Host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells may be cultured in vitro or one or more cells in a non-human animal (e.g., a transgenic animal or a transiently transfected animal).

The methods of the invention may include steps of comparing sequences to each other, including wild-type sequence to one or more mutants. Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, “-”, or “Δ”) in the polymer sequence not containing the inserted or deleted residue.

“Homologous”, in all its grammatical forms and spelling variations, refers to the relationship between two molecules (e.g. proteins, tRNAs, nucleic acids) that possess a “common evolutionary origin”, including proteins from superfamilies in the same species of organism, as well as homologous proteins from different species of organism. Such proteins (and their encoding nucleic acids) have sequence and/or structural homology, as reflected by their sequence similarity, whether in terms of percent identity or by the presence of specific residues or motifs and conserved positions. Homologous molecules frequently also share similar or even identical functions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., Molecular Cloning: A Laboratory Manual, Second Edition (1989) Cold Spring Harbor Laboratory Press, Cold Spring Harbor, N.Y.). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g. 50% formamide, 5× or 6×SSC. SSC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

“Polypeptide,” “peptide” or “protein” are used interchangeably to describe a chain of amino acids that are linked together by chemical bonds called “peptide bonds.” A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

Terms such as “anchor residues,” “fitness,” “fitness contribution,” “dead-end elimination” (DEE), “rotamer,” “rotamer library,” “variable residue position,” “fixed residue position,” “floated,” are defined in US patent application publication US 2004-0053390, incorporated herein by reference.

As used herein, the term “orthogonal” refers to a molecule (e.g., an orthogonal tRNA (O-tRNA) and/or an orthogonal aminoacyl tRNA synthetase (O-RS)) that is used with reduced efficiency (as compared to wild-type or endogenous) by a system of interest (e.g., a translational system, e.g., a cell). Orthogonal refers to the inability or reduced efficiency, e.g., less than 20% efficient, less than 10% efficient, less than 5% efficient, or e.g., less than 1% efficient, of an orthogonal tRNA and/or orthogonal RS to function in the translation system of interest. For example, an orthogonal tRNA in a translation system of interest aminoacylates any endogenous RS of a translation system of interest with reduced or even zero efficiency, when compared to aminoacylation of an endogenous tRNA by the endogenous RS. In another example, an orthogonal RS aminoacylates any endogenous tRNA in the translation system of interest with reduced or even zero efficiency, as compared to aminoacylation of the endogenous tRNA by an endogenous RS. “Improvement in orthogonality” refers to enhanced orthogonality compared to a starting material or a naturally occurring tRNA or RS.

“Wobble degenerate codon” refers to a codon encoding a natural amino acid, which codon, when present in mRNA, is recognized by a natural tRNA anticodon through at least one non-Watson-Crick, or wobble base-pairing (e.g., A-C or G-U base-pairing). Watson-Crick base-pairing refers to either the G-C or A-U (RNA or DNA/RNA hybrid) or A-T (DNA) base-pairing. When used in the context of mRNA codon-tRNA anticodon base-pairing, Watson-Crick base-pairing means all codon-anticodon base-pairings are mediated through either G-C or A-U.

As used herein, proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. For example, any naturally occurring nucleic acid can be modified by any available mutagenesis method to include one or more selector codon. When expressed, this mutagenized nucleic acid encodes a polypeptide comprising one or more non-natural amino acid. The mutation process can, of course, additionally alter one or more standard codon, thereby changing one or more standard amino acid in the resulting mutant protein as well. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein and are generally available.

The term “preferentially aminoacylates” refers to an efficiency, e.g., about 20%, about 30%, about 40%, about 50%, about 60%, about 70%, about 75%, about 85%, about 90%, about 95%, about 99% or more efficient, at which an O-RS aminoacylates an O-tRNA with a non-natural amino acid compared to a naturally occurring tRNA or starting material used to generate the O-tRNA. The non-natural amino acid is then incorporated into a growing polypeptide chain with high fidelity, e.g., at greater than about 20%, 30%, 40%, 50%, 60%, 75%, 80%, 90%, 95%, or greater than about 99% efficiency for a given codon.

The term “complementary” refers to components of an orthogonal pair, O-tRNA and O-RS that can function together, e.g., the O-RS aminoacylates the O-tRNA.

The term “derived from” refers to a component that is isolated from an organism or isolated and modified, or generated, e.g., chemically synthesized, using information of the component from the organism.

The term “translation system” refers to the components necessary to incorporate a naturally occurring or non-natural amino acid into a growing polypeptide chain (protein). For example, components can include ribosomes, tRNA(s), synthetas(es), mRNA and the like. The components of the present invention can be added to a translation system, in vivo or in vitro. An in vivo translation system may be a cell (eukaryotic or prokaryotic cell). An in vitro translation system may be a cell-free system, such as reconstituted one with components from different organisms (purified or recombinantly produced).

The term “inactive RS” refers to a synthetase that have been mutated so that it no longer can aminoacylate its cognate tRNA with an amino acid.

The term “selection agent” refers to an agent that when present allows for a selection of certain components from a population, e.g., an antibiotic, wavelength of light, an antibody, a nutrient or the like. The selection agent can be varied, e.g., such as concentration, intensity, etc.

The term “positive selection marker” refers to a marker than when present, e.g., expressed, activated or the like, results in identification of an organism with the positive selection marker from those without the positive selection marker.

The term “negative selection marker” refers to a marker than when present, e.g., expressed, activated or the like, allows identification of an organism that does not possess the desired property (e.g., as compared to an organism which does possess the desired property).

The term “reporter” refers to a component that can be used to select components described in the present invention. For example, a reporter can include a green fluorescent protein, a firefly luciferase protein, or genes such as β-gal/lacZ (β-galactosidase), Adh (alcohol dehydrogenase) or the like.

The term “not efficiently recognized” refers to an efficiency, e.g., less than about 10%, less than about 5%, or less than about 1%, at which a RS from one organism aminoacylates O-tRNA.

The term “eukaryote” refers to organisms belonging to the phylogenetic domain Eucarya such as animals (e.g., mammals, insects, reptiles, birds, etc.), ciliates, plants, fungi (e.g., yeasts, etc.), flagellates, microsporidia, protists, etc. Additionally, the term “prokaryote” refers to non-eukaryotic organisms belonging to the Eubacteria (e.g., Escherichia coli, Thermus thermophilus, etc.) and Archaea (e.g., Methanococcus jannaschii, Methanobacterium thermoautotrophicum, Halobacterium such as Haloferax volcanii and Halobacterium species NRC-1, A. fulgidus, P. firiosus, P. horikoshii, A. pernix, etc.) phylogenetic domains.

“Normal pH” or “physiological pH” is used in relation to the specific organism and/or application. The normal or physiological pH in human (in vivo) is about pH 7.4-7.6. However, in a different organism, or in a different application (including use in non-biological systems, such as use of enzyme to treat pollution), the “normal pH” might be quite different.

“Small molecule” typically refers to a molecule with a molecular weight of less than 5000 Da. It may include any small peptides, oligonucleotides, lipids, steroids, oligosaccharides, or other drug molecules.

### III. The Genetic Code. Host Cells, and the Degenerate Codons

In one embodiment of the invention, non-natural amino acids may be incorporated into a protein (e.g. Ab, cytokine, insulin, growth hormone, or other signaling molecules, etc.) using the degenerate codon orthogonal system.

The standard genetic code most cells use is well-known in the art, and can be found in numerous textbooks. The genetic code is degenerate, in that the protein biosynthetic machinery utilizes 61 mRNA sense codons to direct the templated polymerization of the 20 natural amino acid monomers. (Crick et al., Nature 192: 1227, 1961). Just two amino acids, i.e., methionine and tryptophan, are encoded by unique mRNA triplets.

The standard genetic code applies to most, but not all, cases. Exceptions have been found in the mitochondrial DNA of many organisms and in the nuclear DNA of a few lower organisms. Some examples are given in the following table.

Examples of non-standard genetic codes.

| | | |
|---|---|---|
| Mitochondria | Vertibrates | UGA→ Trp; AGA, AGG → STOP |
| | Invertibrates | UGA→ Trp; AGA, AGG → Ser |
| | Yeasts | UGA→ Trp; CUN → Thr |
| | Protista | UGA→ Trp; |
| Nucleus | Bacteria | GUG, UUG, AUU, CUG → initiation |
| | Yeasts | CUG → Ser |
| | Ciliates | UAA, UAG → Gln |

*Plant cells use the standard genetic code in both mitochondria and the nucleus.

The NCBI (National Center for Biotechnology Information) maintains a detailed list of the standard genetic code, and genetic codes used in various organisms, including the vertebrate mitochondrial code; the yeast mitochondrial code; the mold, protozoan, and coelenterate mitochondrial code and the mycoplasma/spiroplasma code; the invertebrate mitochondrial code; the ciliate, dasycladacean and hexamita nuclear code; the echinoderm and flatworm mitochondrial code; the euplotid nuclear code; the bacterial and plant plastid code; the alternative yeast nuclear code; the ascidian mitochondrial code; the alternative flatworm mitochondrial code; blepharisma nuclear code; chlorophycean mitochondrial code; trematode mitochondrial code; scenedesmus obliquus mitochondrial code; thraustochytrium mitochondrial code (all incorporated herein by reference). These are primarily based on the reviews by Osawa et al., Microbiol. Rev. 56: 229-264, 1992, and Jukes and Osawa, Comp. Biochem. Physiol. 106B: 489-494, 1993.

Host Cells

The methods of the invention can be practiced within a cell, which enables production levels of proteins to be made for practical purposes. Because of the high degree of conservation of the genetic code and the surrounding molecular machinery, method of the invention can be used in most cells.

In preferred embodiments, the cells used are culturable cells (i.e., cells that can be grown under laboratory conditions). Suitable cells include mammalian cells (human or non-human mammals), bacterial cells, and insect cells, etc.

Degenerate Codon Selection

As described above, all amino acids, with the exception of methionine and tryptophan are encoded by more than one codon. According to the methods of the invention, a codon that is normally used to encode a natural amino acid is reprogrammed to encode an amino acid analog. An amino acid analog can be a naturally occurring or canonical amino acid analog. In a preferred embodiment, the amino acid analog is not a canonically encoded amino acid.

The thermodynamic stability of a codon-anticodon pair can be predicted or determined experimentally. According to the invention, it is preferable that the orthogonal tRNA interacts with the degenerate codon with an affinity (at 37° C.) of at least about 1.0 kcal/mol more strongly, even more preferably 1.5 kcal/mole more strongly, and even more preferably more than 2.0 kcal/mol more strongly than a natural tRNA in the cell would recognize the same sequence. These values are known to one of skill in the art and can be determined by thermal denaturation experiments (see, e.g., Meroueh and Chow, Nucleic Acids Res. 27: 1118, 1999).

The Degenerate Codons

| Amino | | Base- | |
|---|---|---|---|
| Acid | Anticondon | paring | Codon |
| Ala | GGC | W/C¹ | GCC |
| | | Wobble² | GCU |
| | UGC | W/C | GCA |
| | | Wobble | GCG |
| Asp | GUC | W/C | GAC |
| | | Wobble | GAU |
| Asn | GUU | W/C | AAC |
| | | Wobble | AAU |
| Cys | GCA | W/C | UGC |
| | | Wobble | UGU |
| Glu | UUC | W/C | GGA |
| | | Wobble | GAG |
| Gly | GCC | W/C | GGC |
| | | Wobble | GGU |
| His | GUG | W/C | CAC |
| | | Wobble | CAU |
| Ile | GAU | W/C | AUC |
| | | Wobble | AUU |
| Leu | GAG | W/C | CUU |
| | | Wobble | CUC |
| Lys | UUU | W/C | AAA |
| | | Wobble | AAG |
| Phe | GAA | W/C | UUC |
| | | Wobble | UUU |
| Ser | GGA | W/C | UUC |
| | | Wobble | UCU |
| Tyr | GUA | W/C | UAC |
| | | Wobble | UAU |

¹Watson-Crick base pairing,
²Wobble base pairing

When the cell has a single tRNA that recognizes a codon through a perfect complementary interaction between the anticodon of the tRNA and one codon, and recognizes a second, degenerate codon through a wobble or other non-standard base pairing interaction, a new tRNA can be constructed having an anticodon sequence that is perfectly complementary to the degenerate codon.

When the cell has multiple tRNA molecules for a particular amino acid, and one tRNA has an anticodon sequence that is perfectly complementary to the degenerate codon selected, the gene encoding the tRNA can be disabled through any means available to one of skill in the art including, for example, site-directed mutagenesis or deletion of either the gene or the promoter sequence of the gene. Expression of the gene also can be disable through any antisense or RNA interference techniques.

### IV. Non-Natural Amino Acids

Various non-natural amino acids can be used with the methods of the invention for incorporation into the subject antibodies, regardless of the methods used for incorporation. In a preferred embodiment, replacement non-natural amino acids chosen will be sterically similar to the natural amino acids they are designed to replace. The pKa's of such non-natural amino acids are either known to one of skill in the art, or can be determined experimentally using standard biochemical methods well known in the art, or predicted by computational approaches.

For example, histidine side-chain-like moiety, 1,2,4-triazole-3-alanine has pKa's of 3.28 and 10.73, while the actual imidazole moiety of histidine has pKa's of 7.05 and 14.52. On the other hand, the two pKa's of 2-fluoro-histidine are 4.0 and 11.5, respectively. A large number of other histidine analogs can also be used. Examples include: L-methyl histidine, 3-methyl-L-histidine, β-2-thienyl-L-alanine, β-(2-Thiazolyl)-DL-alanine, 1,2,4-triazole-3-alanine, or 2-fluoro-histidine, etc.

However, the useable non-natural amino acids are not limited to those described above. In general, many other non-natural amino acids or their derivatives may be tested or screened to identify suitable side-chain properties. Typically, an in vitro biochemical test may be used as a primary screen to identify candidate non-natural amino acids, such as those with pKa's in the desired ranges. Optionally, a secondary screen may be done to verify these side-chain properties in the context of the incorporated protein, since microenvironments in the protein may possibly alter the side-chain property (e.g. pKs value).

In general, the first step in the protein engineering process is usually to select a set of non-natural amino acids that have the desired chemical properties (e.g. desired pKa value). The selection of non-natural amino acids depends on pre-determined chemical properties one would like to have, and the modifications one would like to make in the target protein. Non-natural amino acids, once selected, can either be purchased from vendors, or chemically synthesized.

A wide variety of non-natural amino acids are available. The non-natural amino acid can generally be chosen based on desired characteristics of the non-natural amino acid, e.g., function of the non-natural amino acid, such as modifying protein biological properties such as toxicity, biodistribution, or half life, structural properties, spectroscopic properties, chemical and/or photochemical properties, catalytic properties, ability to react with other molecules (either covalently or noncovalently), or the like. One of the most important characteristics for regulating pH-sensitive binding is obviously the side-chain pKa value.

As used herein a “non-natural amino acid” refers to any amino acid, modified amino acid, or amino acid analogue other than selenocysteine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. The generic structure of an alpha-amino acid is illustrated by Formula I:

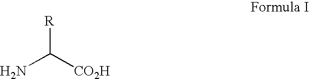

A non-natural amino acid is typically any structure having Formula I wherein the R group is any substituent other than one used in the twenty natural amino acids. See, e.g., any biochemistry text such as Biochemistry by L. Stryer, 3rd ed. 1988, Freeman and Company, New York, for structures of the twenty natural amino acids. Note that, the non-natural amino acids of the present invention may be naturally occurring compounds other than the twenty alpha-amino acids above. Because the non-natural amino acids of the invention typically differ from the natural amino acids in side chain only, the non-natural amino acids form amide bonds with other amino acids, e.g., natural or non-natural, in the same manner in which they are formed in naturally occurring proteins. However, the non-natural amino acids have side chain groups that distinguish them from the natural amino acids. For example, R in Formula I optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-natural amino acids of interest include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto containing amino acids, amino acids comprising polyethylene glycol or polyether, heavy atom substituted amino acids, chemically cleavable and/or photocleavable amino acids, amino acids with an elongated side chains as compared to natural amino acids, e.g., polyethers or long chain hydrocarbons, e.g., greater than about 5 or greater than about 10 carbons, carbon-linked sugar-containing amino acids, redox-active amino acids, amino thioacid containing amino acids, and amino acids comprising one or more toxic moiety.

In addition to non-natural amino acids that contain novel side chains, non-natural amino acids also optionally comprise modified backbone structures, e.g., as illustrated by the structures of Formula II and III:

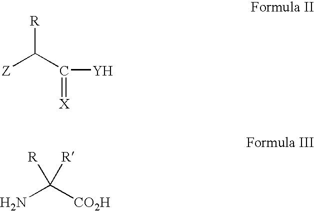

wherein Z typically comprises OH, NH₂, SH, NH—R′, or S—R′; X and Y, which may be the same or different, typically comprise S or O, and R and R′, which are optionally the same or different, are typically selected from the same list of constituents for the R group described above for the non-natural amino acids having Formula I as well as hydrogen. For example, non-natural amino acids of the invention optionally comprise substitutions in the amino or carboxyl group as illustrated by Formulas II and III. Non-natural amino acids of this type include, but are not limited to, α-hydroxy acids, α-thioacids α-aminothiocarboxylates, e.g., with side chains corresponding to the common twenty natural amino acids or non-natural side chains. In addition, substitutions at the α-carbon optionally include L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, β and γ amino acids such as substituted β-alanine and γ-amino butyric acid.

For example, many non-natural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C6-C20 straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, α-hydroxy derivatives, β-substituted derivatives, cyclic derivatives, and amide substituted glutamine derivatives. Example phenylalanine analogs include, but are not limited to, meta-substituted phenylalanines, wherein the substituent comprises a hydroxy group, a methoxy group, a methyl group, an allyl group, an acetyl group, or the like.

Specific examples of non-natural amino acids include, but are not limited to, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like. The structures of a variety of non-limiting non-natural amino acids are provided in the figures, e.g., FIGS. 29, 30, and 31 of US 2003/0108885 A1 (entire content incorporated herein by reference).

Typically, the non-natural amino acids of the invention are selected or designed to provide additional characteristics unavailable in the twenty natural amino acids. For example, non-natural amino acid are optionally designed or selected to modify the biological properties of a protein, e.g., into which they are incorporated. For example, the following properties are optionally modified by inclusion of an non-natural amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like.

Further details regarding non-natural amino acids are described in US 2003-0082575 A1, entitled “In vivo Incorporation of Non-natural Amino Acids,” filed on Apr. 19, 2002, which is incorporated herein by reference.

Additionally, other examples optionally include (but are not limited to) a non-natural analogue of a tyrosine amino acid; a non-natural analogue of a glutamine amino acid; a non-natural analogue of a phenylalanine amino acid; a non-natural analogue of a serine amino acid; a non-natural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged amino acid; a photoisomerizable amino acid; a biotin or biotin-analogue containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

Many of the non-natural amino acids provided above are commercially available, e.g., from Sigma (USA) or Aldrich (Milwaukee, Wis., USA). Those that are not commercially available are optionally synthesized as provided in the examples of US 2004/138106 A1 (incorporated herein by reference) or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). See also WO 02/085923 for additional synthesis of non-natural amino acids.

For example, meta-substituted phenylalanines are synthesized in a procedure as outlined in WO 02/085923 (see, e.g., FIG. 14 of the publication). Typically, NBS (N-bromosuccinimide) is added to a meta-substituted methylbenzene compound to give a meta-substituted benzyl bromide, which is then reacted with a malonate compound to give the meta substituted phenylalanine. Typical substituents used for the meta position include, but are not limited to, ketones, methoxy groups, alkyls, acetyls, and the like. For example, 3-acetyl-phenylalanine is made by reacting NBS with a solution of 3-methylacetophenone. For more details see the examples below. A similar synthesis is used to produce a 3-methoxy phenylalanine. The R group on the meta position of the benzyl bromide in that case is —OCH₃. See, e.g., Matsoukas et al., J. Med. Chem., 1995, 38, 4660-4669.

In some embodiments, the design of non-natural amino acids is biased by known information about the active sites of synthetases, e.g., orthogonal tRNA synthetases used to aminoacylate an orthogonal tRNA. For example, three classes of glutamine analogs are provided, including derivatives substituted at the nitrogen of amide (1), a methyl group at the γ-position (2), and a N-Cγ-cyclic derivative (3). Based upon the x-ray crystal structure of E. coli GlnRS, in which the key binding site residues are homologous to yeast GlnRS, the analogs were designed to complement an array of side chain mutations of residues within a 10 shell of the side chain of glutamine, e.g. a mutation of the active site Phe233 to a small hydrophobic amino acid might be complemented by increased steric bulk at the Cγ position of Gln.

For example, N-phthaloyl-L-glutamic 1,5-anhydride (compound number 4 in FIG. 23 of WO 02/085923) is optionally used to synthesize glutamine analogs with substituents at the nitrogen of the amide. See, e.g., King & Kidd, A New Synthesis of Glutamine and of γ-Dipeptides of Glutamic Acid from Phthylated Intermediates. J. Chem. Soc., 3315-3319, 1949; Friedman & Chatterrji, Synthesis of Derivatives of Glutamine as Model Substrates for Anti-Tumor Agents. J. Am. Chem. Soc. 81, 3750-3752, 1959; Craig et al., Absolute Configuration of the Enantiomers of 7-Chloro-4[[4-(diethylamino)-1-methylbutyl]amino]quinoline (Chloroquine). J. Org. Chem. 53, 1167-1170, 1988; and Azoulay et al., Glutamine analogues as Potential Antimalarials, Eur. J. Med. Chem. 26, 201-5, 1991. The anhydride is typically prepared from glutamic acid by first protection of the amine as the phthalimide followed by refluxing in acetic acid. The anhydride is then opened with a number of amines, resulting in a range of substituents at the amide. Deprotection of the phthaloyl group with hydrazine affords a free amino acid as shown in FIG. 23 of WO 2002/085923.

Substitution at the γ-position is typically accomplished via alkylation of glutamic acid. See, e.g., Koskinen & Rapoport, Synthesis of 4-Substituted Prolines as Conformationally Constrained Amino Acid Analogues. J. Org. Chem. 54, 1859-1866, 1989. A protected amino acid, e.g., as illustrated by compound number 5 in FIG. 24 of WO 02/085923, is optionally prepared by first alkylation of the amino moiety with 9-bromo-9-phenylfluorene (PhflBr) (see, e.g., Christie & Rapoport, Synthesis of Optically Pure Pipecolates from L-Asparagine. Application to the Total Synthesis of (+)-Apovincamine through Amino Acid Decarbonylation and Iminium Ion Cyclization. J. Org. Chem. 1989, 1859-1866, 1985) and then esterification of the acid moiety using O-tert-butyl-N,N′-diisopropylisourea. Addition of KN(Si(CH₃)₃)₂ regioselectively deprotonates at the α-position of the methyl ester to form the enolate, which is then optionally alkylated with a range of alkyl iodides. Hydrolysis of the t-butyl ester and Phfl group gave the desired γ-methyl glutamine analog (Compound number 2 in FIG. 24 of WO 02/085923).

An N-Cγ cyclic analog, as illustrated by Compound number 3 in FIG. 25 of WO 02/085923, is optionally prepared in 4 steps from Boc-Asp-Ot-Bu as previously described. See, e.g., Barton et al., Synthesis of Novel α-Amino-Acids and Derivatives Using Radical Chemistry: Synthesis of L- and D-α-Amino-Adipic Acids, L-α-aminopimelic Acid and Appropriate Unsaturated Derivatives. Tetrahedron Lett. 43, 4297-4308, 1987, and Subasinghe et al., Quisqualic acid analogues: synthesis of beta-heterocyclic 2-aminopropanoic acid derivatives and their activity at a novel quisqualate-sensitized site. J. Med. Chem. 35 4602-7, 1992. Generation of the anion of the N-t-Boc-pyrrolidinone, pyrrolidinone, or oxazolidone followed by the addition of the compound 7, as shown in FIG. 25, results in a Michael addition product. Deprotection with TFA then results in the free amino acids.

In addition to the above non-natural amino acids, a library of tyrosine analogs has also been designed. Based upon the crystal structure of B. stearothermophilus TyrRS, whose active site is highly homologous to that of the M. jannashii synthetase, residues within a 10 shell of the aromatic side chain of tyrosine were mutated (Y32, G34, L65, Q155, D158, A167, Y32 and D158). The library of tyrosine analogs, as shown in FIG. 26 of WO 02/085923, has been designed to complement an array of substitutions to these active site amino acids. These include a variety of phenyl substitution patterns, which offer different hydrophobic and hydrogen-bonding properties. Tyrosine analogs are optionally prepared using the general strategy illustrated by WO 02/085923 (see, e.g., FIG. 27 of the publication). For example, an enolate of diethyl acetamidomalonate is optionally generated using sodium ethoxide. A desired tyrosine analog can then be prepared by adding an appropriate benzyl bromide followed by hydrolysis.

Many biosynthetic pathways already exist in cells for the production of amino acids and other compounds. While a biosynthetic method for a particular non-natural amino acid may not exist in nature, e.g., in E. coli, the invention provide such methods. For example, biosynthetic pathways for non-natural amino acids are optionally generated in E. coli by adding new enzymes or modifying existing E. coli pathways. Additional new enzymes are optionally naturally occurring enzymes or artificially evolved enzymes. For example, the biosynthesis of p-aminophenylalanine (as presented, e.g., in WO 02/085923) relies on the addition of a combination of known enzymes from other organisms. The genes for these enzymes can be introduced into a cell, e.g., an E. coli cell, by transforming the cell with a plasmid comprising the genes. The genes, when expressed in the cell, provide an enzymatic pathway to synthesize the desired compound. Examples of the types of enzymes that are optionally added are provided in the examples below. Additional enzymes sequences are found, e.g., in Genbank. Artificially evolved enzymes are also optionally added into a cell in the same manner. In this manner, the cellular machinery and resources of a cell are manipulated to produce non-natural amino acids.

A variety of methods are available for producing novel enzymes for use in biosynthetic pathways or for evolution of existing pathways. For example, recursive recombination, e.g., as developed by Maxygen, Inc., is optionally used to develop novel enzymes and pathways. See, e.g., Stemmer 1994, “Rapid evolution of a protein in vitro by DNA shuffling,” Nature 370(4): 389-391; and Stemmer, 1994, “DNA shuffling by random fragmentation and reassembly: In vitro recombination for molecular evolution,” Proc. Natl. Acad. Sci. USA. 91: 10747-10751. Similarly DesignPath™, developed by Genencor is optionally used for metabolic pathway engineering, e.g., to engineer a pathway to create a non-natural amino acid in E. coli. This technology reconstructs existing pathways in host organisms using a combination of new genes, e.g., identified through functional genomics, and molecular evolution and design. Diversa Corporation also provides technology for rapidly screening libraries of genes and gene pathways, e.g., to create new pathways.

Typically, the biosynthesis methods of the invention, e.g., the pathway to create p-aminophenylalanine (pAF) from chorismate, do not affect the concentration of other amino acids produced in the cell. For example a pathway used to produce pAF from chorismate produces pAF in the cell while the concentrations of other aromatic amino acids typically produced from chorismate are not substantially affected. Typically the non-natural amino acid produced with an engineered biosynthetic pathway of the invention is produced in a concentration sufficient for efficient protein biosynthesis, e.g., a natural cellular amount, but not to such a degree as to affect the concentration of the other amino acids or exhaust cellular resources. Typical concentrations produced in vivo in this manner are about 10 mM to about 0.05 mM. Once a bacterium is transformed with a plasmid comprising the genes used to produce enzymes desired for a specific pathway and a twenty-first amino acid, e.g., pAF, dopa, O-methyl-L-tyrosine, or the like, is generated, in vivo selections are optionally used to further optimize the production of the non-natural amino acid for both ribosomal protein synthesis and cell growth.

### V. Aminoacyl-tRNA Synthetases

The aminoacyl-tRNA synthetase (used interchangeably herein with AARS or “synthetase”) used in certain embodiments of the invention (e.g. the degenerate codon orthogonal system) can be a naturally occurring synthetase derived from a different organism, a mutated synthetase, or a designed synthetase.

The synthetase used can recognize the desired (non-natural) amino acid analog selectively over related amino acids available to the cell. For example, when the amino acid analog to be used is structurally related to a naturally occurring amino acid in the cell, the synthetase should charge the orthogonal tRNA molecule with the desired amino acid analog with an efficiency at least substantially equivalent to that of, and more preferably at least about twice, 3 times, 4 times, 5 times or more than that of the naturally occurring amino acid. However, in cases in which a well-defined protein product is not necessary, the synthetase can have relaxed specificity for charging amino acids. In such an embodiment, a mixture of orthogonal tRNAs could be produced, with various amino acids or analogs.

In certain embodiments, it is preferable that the synthetase have activity both for the amino acid analog and for the amino acid that is encoded by the degenerate codon of the orthologous tRNA molecule. In the absence of the amino acid analog, this allows the cell to continue to grow, while upon addition of the amino acid analog to the cell, allows a switch to allow incorporation of the amino acid analog. The synthetase also should be relatively specific for the orthogonal tRNA molecule over other naturally occurring tRNA molecules within the cell. Choosing a tRNA-synthetase pair from an unrelated organism will generally allow for such selectivity. The selectivity of the synthetase for the orthogonal tRNA can be tested experimentally by testing the ability of the orthogonal synthetase to charge the natural tRNAs of the host cell with canonical amino acids. (Orthogonality could be confirmed by even natural amino acids, because tRNA recognition domain in synthetase might be different from that for amino acid analogs. Of course, amino acid analogs should be charged only into orthogonal tRNA efficiently by synthetase, after binding site of synthetase is appropriately modified). Such procedures are described, for example, in Doctor and Mudd, J. Biol. Chem. 238: 3677-3681, 1963; Wang et al., Science 292: 498-500, 2001).

The method involves introduction into the host cell of a heterologous aminoacyl-tRNA synthetase and its cognate tRNA. If cross-charging between the heterologous pair and the translational apparatus of the host is slow or absent, and if the analogue is charged only by the heterologous synthetase, insertion of the analog can be restricted (or at least biased) to sites characterized by the most productive base-pairing between the heterologous tRNA and the messenger RNA of interest.

A synthetase can be obtained by a variety of techniques known to one of skill in the art, including combinations of such techniques as, for example, computational methods, selection methods, and incorporation of synthetases from other organisms (see below).

In certain embodiments, synthetases can be used or developed that efficiently charge tRNA molecules that are not charged by synthetases of the host cell. For example, suitable pairs may be generally developed through modification of synthetases from organisms distinct from the host cell.

In certain embodiments, the synthetase can be developed by selection procedures.

In certain embodiments, the synthetase can be designed using computational techniques such as those described in Datta et al., J. Am. Chem. Soc. 124: 5652-5653, 2002, and in co-pending U.S. patent application Ser. No. 10/375,298 (or US patent application publication US20040053390A1, entire content incorporated herein by reference).

Specifically, in one embodiment, the subject method partly depends on the design and engineering of natural AARS to a modified form that has relaxed substrate specificity, such that it can uptake non-canonical amino acid analogs as a substrate, and charge a modified tRNA (with its anticodon changed) with such a non-canonical amino acid. The following sections briefly describe a method for the generation of such modified AARS, which method is described in more detail in US patent application publication US20040053390A1, the entire contents of which are incorporated herein by reference.

Briefly, the methods described therein relate to computational tools for modifying the substrate specificity of an AminoAcyl tRNA Synthetases (AARSs) through mutation to enable the enzyme to more efficiently utilize amino acid analog(s) in protein translation systems, either in vitro or in whole cells. A salient feature to the described invention is methods and tools for systematically redesigning the substrate binding site of an AARS enzyme to facilitate the use of non-natural substrates in the peptide or protein translation reaction the enzyme catalyzes.

According to the method, a rotamer library for the artificial amino acid is built by varying its torsional angles to create rotamers that would fit in the binding pocket for the natural substrate. The geometric orientation of the backbone of the amino acid analog is specified by the crystallographic orientation of the backbone of the natural substrate in the crystal structure. Amino acids in the binding pocket of the synthetase that interact with the side chain on the analog are allowed to vary in identity and rotameric conformation in the subsequent protein design calculations.

The protocol also employ a computational method to enhance the interactions between the substrate and the protein positions. This is done by scaling up the pair-wise energies between the substrate and the amino acids allowed at the design positions on the protein in the energy calculations. In an optimization calculation where the protein-substrate interactions are scaled up compared to the intra-protein interactions, sequence selection is biased toward selecting amino acids to be those that have favorable interaction with the substrate.

The described method helped to construct a new modified form of the E. coli phenylalanyl-tRNA synthetase, based on the known structure of the related Thermus thermophilus PheRS (tPheRS). The new modified form of the E. coli phenylalanyl-tRNA synthetase (ePheRS**) allows efficient in vivo incorporation of reactive aryl ketone functionality into recombinant proteins. The results described therein also demonstrate the general power of computational protein design in the development of aminoacyl-tRNA synthetases for activation and charging of non-natural amino acids.

In certain embodiments, the orthogonal tRNA/synthetase pair is generated by importing a tRNA/synthetase pair from another organism into the translation system of interest, such as Escherichia coli or yeast. In this E. coli example, the properties of the heterologous synthetase candidate include, e.g., that it does not charge any Escherichia coli tRNA, and the properties of the heterologous tRNA candidate include, e.g., that it is not acylated by any Escherichia coli synthetase. In addition, the O-tRNA derived therefrom is orthogonal to all Escherichia coli synthetases.

Using the methods of the present invention, the pairs and components of pairs desired above are evolved to generate orthogonal tRNA/synthetase pairs that possess desired characteristic, e.g., that can preferentially aminoacylate an O-tRNA with a non-natural amino acid.

In certain embodiments, the O-tRNA and the O-RS can be derived by mutation of a naturally occurring tRNA and RS from a variety of organisms. In one embodiment, the O-tRNA and O-RS are derived from at least one organism, where the organism is a prokaryotic organism, e.g., Methanococcus jannaschii, Methanobacterium thermoautotrophicum, Halobacterium, Escherichia coli, A. fulgidus, P. furiosus, P. horikoshii, A. pernix, T. thermophilus, or the like. Optionally, the organism is a eukaryotic organism, e.g., plants (e.g., complex plants such as monocots, or dicots), algea, fungi (e.g., yeast, etc), animals (e.g., mammals, insects, arthropods, etc.), insects, protists, or the like. Optionally, the O-tRNA is derived by mutation of a naturally occurring tRNA from a first organism and the O-RS is derived by mutation of a naturally occurring RS from a second organism. In one embodiment, the O-tRNA and O-RS can be derived from a mutated tRNA and mutated RS. In certain embodiments, the O-RS and O-tRNA pair from a first organism is provided to a translational system of a second organism, which optionally has non-functional endogenous RS/tRNA pair with respect to the codons recognized by the O-tRNA.

The O-tRNA and the O-RS also can optionally be isolated from a variety of organisms. In one embodiment, the O-tRNA and O-RS are isolated from at least one organism, where the organism is a prokaryotic organism, e.g., Methanococcus jannaschii, Methanobacterium thermoautotrophicum, Halobacterium, Escherichia coli, A. fulgidus, P. furiosus, P. horikoshii, A. pernix, T. thermophilus, or the like. Optionally, the organism is a eukaryotic organism, e.g., plants (e.g., complex plants such as monocots, or dicots), algea, fungi (e.g., yeast, etc), animals (e.g., mammals, insects, arthropods, etc.), insects, protists, or the like. Optionally, the O-tRNA is isolated from a naturally occurring tRNA from a first organism and the O-RS is isolated from a naturally occurring RS from a second organism. In one embodiment, the O-tRNA and O-RS can be isolated from one or more library (which optionally comprises one or more O-tRNA and/or O-RS from one or more organism (including those comprising prokaryotes and/or eukaryotes).

The orthogonal tRNA-RS pair, e.g., derived from at least a first organism or at least two organisms, which can be the same or different, can be used in a variety of organisms, e.g., a second organism. The first and the second organisms of the methods of the present invention can be the same or different. As described above, the individual components of a pair can be derived from the same organism or different organisms. For example, tRNA can be derived from a prokaryotic organism, e.g., an archaebacterium, such as Methanococcus jannaschii and Halobacterium NRC-1 or a eubacterium, such as Escherichia coli, while the synthetase can be derived from same or another prokaryotic organism, such as, Methanococcus jannaschii, Archaeoglobus fulgidus, Methanobacterium thermoautotrophicum, P. furiosus, P. horikoshii, A. pernix, T. thermophilus, Halobacterium, Escherichia coli or the like. Eukaryotic sources can also be used, e.g., plants (e.g., complex plants such as monocots, or dicots), algae, protists, fungi (e.g., yeast, etc.), animals (e.g., mammals, insects, arthropods, etc.), or the like.

Methods for selecting an orthogonal tRNA-tRNA synthetase pair for use in an in vivo translation system of a second organism are also included in the present invention. The methods include: introducing a marker gene, a tRNA and an aminoacyl-tRNA synthetase (RS) isolated or derived from a first organism into a first set of cells from the second organism; introducing the marker gene and the tRNA into a duplicate cell set from the second organism; and, selecting for surviving cells in the first set that fail to survive in the duplicate cell set, where the first set and the duplicate cell set are grown in the presence of a selection agent, and where the surviving cells comprise the orthogonal tRNA-tRNA synthetase pair for use in the in the in vivo translation system of the second organism. In one embodiment, comparing and selecting includes an in vivo complementation assay. In another embodiment, the concentration of the selection agent is varied. The same assay may also be conducted in an in vitro system based on the second organism.

The AARS may also be generated by mutagenesis and selection/screening. See US20040053390A1, incorporated by reference.

### VI. Nucleic Acid and Polypeptide Sequence Variants

As described herein, the invention provides for nucleic acid polynucleotide sequences and polypeptide amino acid sequences, e.g., O-tRNAs and O-RSs (and their coding polynucleotides thereof), polynucleotide sequences containing (selected) degenerate codon mutations designed for incorporating non-natural amino acids at such codon locations using the degenerate codon orthogonal system, and, e.g., compositions and methods comprising said sequences. Examples of said sequences, e.g., O-tRNAs and O-RSs are disclosed herein. However, one of skill in the art will appreciate that the invention is not limited to those sequences disclosed herein. One of skill will appreciate that the present invention also provides many related and unrelated sequences with the functions described herein, e.g., encoding an O-tRNA or an O-RS.

One of skill will also appreciate that many variants of the disclosed sequences are included in the invention. For example, conservative variations of the disclosed sequences that yield a functionally identical sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g. standard sequence comparison techniques, are also included in the invention. In the case of incorporating non-natural amino acids by degenerate codon orthogonal system, the selectively mutated codons for non-natural amino acids are not changed in such variant sequences. Neither are new mutations generated for additional non-natural amino acid incorporation sites.

### VII. Exemplary Uses

Well over 100 non-coded amino acids (all ribosomally acceptable) have been reportedly introduced into proteins using other methods (see, for example, Schultz et al., J. Am. Chem. Soc., 103: 1563-1567, 1981; Hinsberg et al., J. Am. Chem. Soc., 104: 766-773, 1982; Pollack et al., Science, 242: 1038-1040, 1988; Nowak et al., Science, 268: 439-442, 1995) all these analogs may be used in the subject methods for efficient incorporation of these analogs into protein products. In general, the method of the instant invention can be used to incorporate amino acid analogs into protein products either in vitro or in vivo.

In another preferred embodiment, two or more analogs may be used in the same in vitro or in vivo translation system, each with its O-tRNA/O-RS pairs. This is more easily accomplished when a natural amino acid is encoded by four or more codons (such as six for Leu and Arg). However, for amino acids encoded by only two codons, one can be reserved for the natural amino acid, while the other “shared” by one or more amino acid analog(s). These analogs may resemble only one natural amino acid (for example, different Phe analogs), or resemble different amino acids (for example, analogs of Phe and Tyr).

For in vitro use, one or more O-RSs of the instant invention can be recombinantly produced and supplied to any the available in vitro translation systems (such as the commercially available Wheat Germ Lysate-based PROTEINscript-PRO™, Ambion's E. coli system for coupled in vitro transcription/translation; or the rabbit reticulocyte lysate-based Retic Lysate IVT™ Kit from Ambion). Optionally, the in vitro translation system can be selectively depleted of one or more natural AARSs (by, for example, immunodepletion using immobilized antibodies against natural AARS) and/or natural amino acids so that enhanced incorporation of the analog can be achieved. Alternatively, nucleic acids encoding the re-designed O-RSs may be supplied in place of recombinantly produced AARSs. The in vitro translation system is also supplied with the analogs to be incorporated into mature protein products.

Although in vitro protein synthesis usually cannot be carried out on the same scale as in vivo synthesis, in vitro methods can yield hundreds of micrograms of purified protein containing amino acid analogs. Such proteins have been produced in quantities sufficient for their characterization using circular dichroism (CD), nuclear magnetic resonance (NMR) spectrometry, and X-ray crystallography. This methodology can also be used to investigate the role of hydrophobicity, packing, side chain entropy and hydrogen bonding in determining protein stability and folding. It can also be used to probe catalytic mechanism, signal transduction and electron transfer in proteins. In addition, the properties of proteins can be modified using this methodology. For example, photocaged proteins can be generated that can be activated by photolysis, and novel chemical handles have been introduced into proteins for the site specific incorporation of optical and other spectroscopic probes.

The development of a general approach for the incorporation of amino acid analogs into proteins in vivo, directly from the growth media, would greatly enhance the power of non-natural amino acid mutagenesis. For the purpose of the instant invention, non-natural amino acids with desirable side-chain pKa values may be selectively incorporated to modulated pH-sensitive binding.

For in vivo use, one or more AARS of the instant invention can be supplied to a host cell (prokaryotic or eukaryotic) as genetic materials, such as coding sequences on plasmids or viral vectors, which may optionally integrate into the host genome and constitutively or inducibly express the re-designed AARSs. A heterologous or endogenous protein of interest can be expressed in such a host cell, at the presence of supplied amino acid analogs. The protein products can then be purified using any art-recognized protein purification techniques, or techniques specially designed for the protein of interest.

The above described uses are merely a few possible means for generating a transcript which encodes a polypeptide. In general, any means known in the art for generating transcripts can be employed to synthesize proteins with amino acid analogs. For example, any in vitro transcription system or coupled transcription/translation systems can be used for generate a transcript of interest, which then serves as a template for protein synthesis. Alternatively, any cell, engineered cell/cell line, or functional components (lysates, membrane fractions, etc.) that is capable of expressing proteins from genetic materials can be used to generate a transcript. These means for generating a transcript will typically include such components as RNA polymerase (T7, SP6, etc.) and co-factors, nucleotides (ATP, CTP, GTP, UTP), necessary transcription factors, and appropriate buffer conditions, as well as at least one suitable DNA template, but other components may also added for optimized reaction condition. A skilled artisan would readily envision other embodiments similar to those described herein.

The following section describes a few specific uses of the instant methods and systems for non-natural amino acid incorporation. These are meant to be illustrative and by no means limiting in any respect.

### A. Enhance Half-Life of Cytokines and Growth Factors Through Increased Recycling:

Besides clearance through kidneys and the liver, a significant proportion of biotherapeutics are cleared through receptor-mediated degradation. Cytokines and growth factors, when bound to their receptors, are internalized into cellular compartments called endosomes where the receptor-ligand complexes are degraded. However, those ligands that dissociate rapidly from their receptors in the endosome are recycled back to the cell surface and avoid depletion, thereby eliciting increased half-life. For general background, see Endocytosis, Edited by Ira Pastan and Mark C. Willingham, Plenum Press, N.Y., 1985.

Therefore, non-natural amino acids may be incorporated into insulin (or other signaling molecules that may be down-regulated via receptor-mediated endocytosis or cell-based clearance mechanisms), such that its pH-sensitive binding may be modulated, resulting in early/faster release of the signaling molecule from its receptor complex in endosome. The insulin (or signaling molecules) may then be preferentially recycled back to the cell surface, where it may bind another receptor and initiate another round of signaling, thus effectively resulting in longer half-life of the signaling molecules. In this embodiment, the higher pKa of the non-natural amino acids results in the dissociation of the signaling molecule and its receptor at a relatively higher pH (e.g. about 0.5-1.5 units higher) in an early endosome.

Sarkar et al. reported an approach to use natural amino acids to design a variant of G-CSF, which has reduced binding affinity for its receptor in the endosome, thus achieving a half-life of 500 hours, compared to only about 50 hours for unmodified GSCF (Sarkar et al., Nature Biotechnology 20, 908-913, 2002). Specifically, Sarkar et al. used computationally predicted histidine substitutions that switch protonation states between cell-surface and endosomal pH. Molecular modeling of binding electrostatics (incorporated herein by reference for the same use of testing incorporated non-natural amino acids in the instant methods) indicates two different single-histidine mutants that fulfill the design requirements. Experimental assays demonstrate that each mutant indeed exhibits an order-of-magnitude increase in medium half-life along with enhanced potency due to increased endocytic recycling.

However, as described above, chemistries offered by natural amino acids to modulate the binding process are limited in number and scope. In contrast, non-natural amino acids will offer a significantly better spectrum of useful chemistries, and thus more control on ligand-receptor binding affinities. Such improvements will exhibit more efficient ligand recycling, leading to increase in ligand half-life by orders of magnitudes. This method for designing cytokines and growth factors that exhibit reduced receptor-mediated degradation will be very useful in providing an alternative strategy for increasing half-life of those molecules that are not amenable to other methods, such as PEGylation.

Thus the instant invention provides a method to incorporate non-natural amino acids, the unique chemistries of which can be leveraged for designing the next generation of cytokines and growth factors (or any other signaling molecules regulated by receptor-mediated endocytosis) that maintain high binding affinities for receptors on the cell surface, while having significantly lower binding affinities once they are internalized.

The instant invention can be used to incorporate non-natural amino acid(s) into a number of protein therapeutics, such as the recombinant Cerezyme® and increase their half-lives without substantially lose its intended bioactivity, thus significantly reduce the amount of proteins needed per patient in a given amount of treatment period. This will reduce the cost and/or increase profit margin, resulting in a cheaper, if not better therapeutics that is more affordable.

Numerous other proteins go through receptor-mediated endocytosis, including: toxins or lectins selected from: Diptheria Toxins, Pseudomonas toxins, Cholera toxins, Ricins, or Concanavalin A; viruses selected from: Rous sarcoma virus, Semliki forest virus, Vesicular stomatitis virus, or Adenovirus; serum transport proteins selected from: Transferrin, Low density lipoprotein, Transcobalamin, or Yolk protein; antibodies selected from: IgE, Polymeric IgA, Maternal IgG, or IgG (via Fc receptors); or hormones or growth factors selected from: insulin, EGF, Growth Hormone, Thyroid stimulating hormone, NGF, Calcitonin, Glucagon, Prolactin, Luteinizing Hormone, Thyroid hormone, PDGF, Interferon, or Catecholamine.

When the ligand binds to its specific receptor, the ligand-receptor complex accumulates in the so-called coated pits, which pre-concentrates in one area of a cell, and eventually is internalized through endocytosis. After entering the cytoplasm, the endocytotic vesicle loses its clathrin coat, and quickly fuses with other such vesicles in a process called “homotypic” (same type) fusion. Markers for early endosomes include pH of around 5.9-6.0.

Early endosome can release the ligand from the receptor complex. The receptor may be recycled to the surface by vesicles that bud from the endosome and then target the plasma membrane. After these recycling vesicles fuse with the plasma membrane, the receptor is returned to the cell surface for further binding and activity. Then, the early endosome converts to a late endosome, which has a more acidic environment (pH of about 5.0-6.0).

The exact fate of the receptor in the membrane appears to vary with the cell. It can also be degraded. However, some receptors move to the Golgi complex to be added back to membranes in the Trans Golgi region. This would recycle the receptor. This process is similar to the process by which lysosomal enzyme receptors are recycled. In many cases, the receptor is sent back to the plasma membrane after a transport vesicle buds from the endosome. The endocytic recyclin compartment generally has a pH of about 6.4-6.5.

Late endosomes are formed as the pH continues to drop to 5.0-6.0. Also, clathrin-coated vesicles from the Trans Golgi Network carry digestive enzymes to the late endosome and fuse with these structures, releasing their contents. The late endosome thus becomes a degradative body. They function to degrade many proteins and lipids. They also are responsible for returning the MPR receptors back to the Trans Golgi network. They recycle these by budding off membranes that carry back the receptors and target the Trans Golgi membranes for fusion. After fusion, the MRP receptors are available to capture and sort new degradative enzymes for future trafficking to the late endosomes.

Finally, late endosomes may not be able to digest all the material. Therefore, the next step is a fusion of late endosomes and lysosomes (compartment pH generally about 5.0-5.5), creating a hybrid organelle. Residual heavily glycosylated lysosomal associated membrane proteins (LAMPs) may thus be transmitted to lysosomes. LAMPs then become a marker for a late endosome or a lysosome. Since lysosomes do not have MPR receptors (they have all been sent to the Golgi), one could distinguish the lysosome and the late endosome on the basis of labeling for MPR. Thus, fusion begins after the MPR have been sent back to the Trans Golgi.

Thus if a protein ligand modified by a non-natural amino acid can be dissociated from its receptor at around the pH present in the endocytic recycling compartment (about pH 6.4-6.5), the protein ligand may be preferentially recycled back to cell surface via this compartment, rather than going through the late endosome—lysosome pathway for degredation.

### B. (Multi-)Drug Immunoconjugates

The global market for monoclonal antibody therapeutics reached a total of $7.2 billion in 2003. The market has been growing at an impressive compound average annual growth rate of 53% over the previous five years, and is estimated to reach US$26 billion by the end of the decade (average annual growth rate of 18%).

More than 270 industry antibody R&D projects related to cancer therapy have been identified. Among them, there are almost 100 industry related R&D projects utilizing conjugated antibodies as a therapeutic strategy, some are already in different phases of clinical development (see Monoclonal Antibody Therapeutics: Current Market Dynamics & Future Outlook, Research and Markets Ltd, 2004; Improved Monoclonals on the Rise, Research and Markets Ltd, 2004; Anticancer Monoclonal Antibody Database, Bioportfolio, 2003).

Immunoconjugation may be used to increase the therapeutic efficacies of antibodies. However, current technologies allow attachment of only a single type of drug to an antibody. This is primarily due to the limitations in the scope of chemistries available in the set of natural amino acids, which do not allow precise control over the immunoconjugation processes.

Attempts to attach multiple drugs on an antibody using current technologies lead to significant heterogeneity from molecule to molecule, and inconsistencies from lot to lot. This is far from ideal in the context of tumor therapies, since the best strategy to treat tumors is frequently through using cocktails of drugs.

Non-natural amino acids can be used to provide a wide variety of new chemistries to attach drugs site-specifically, thus enabling the provision of tumor-targeted, multi-drug regimens to cancer patients. For example, the instant methods can be used to produce immunoconjugates either by attaching a single type of drug site-specifically on to antibodies and antibody fragments to overcome issues related to heterogeneity, or by attaching multiple drug-types site-specifically on to antibodies and antibody fragments in a stoichiometrically controlled manner. In other words, the methods of the instant invention can be used to design a novel class of immunoconjugates that carry a combination of drugs that can be delivered simultaneously and specifically to the tumor, where the therapeutic molecules in the medicament are highly homogeneous, with lot to lot consistency. The major advantages of such immunoconjugates include:

Simultaneous targeted delivery of multiple drugs that act synergistically in killing tumor cells

Combining drugs that act in different phases of the cell cycle to increase the number of cells exposed to cytotoxic effects

Focused delivery of the cytotoxic agents to tumor cells maximizing its antitumor effect

Minimized exposure to normal tissue

Precise control over drug payloads and drug ratios leading to homogenous final products

For example, EP0328147B1 describes novel immunoconjugates, methods for their production, pharmaceutical compositions and method for delivering cytotoxic anthracyclines to a selected population of cells desired to be eliminated. More particularly, the invention relates to immunoconjugates comprised of an antibody reactive with a selected cell population to be eliminated, the antibody having a number of cytotoxic anthracycline molecules covalently linked to its structure. Each anthracycline molecule is conjugated to the antibody via a linker arm, the anthracycline being bound to that linker via an acid-sensitive acylhydrazone bond at the 13-keto position of the anthracycline. A preferred embodiment of the invention relates to an adriamycin immunoconjugate wherein adriamycin is attached to the linker arm through an acylhydrazone bond at the 13-keto position. The linker additionally contains a disulfide or thioether linkage as part of the antibody attachment to the immunoconjugate. The immunoconjugates and methods of the invention are useful in antibody-mediated drug delivery systems for the preferential killing of a selected cell population in the treatment of diseases such as cancers and other tumors, non-cytocidal viral or other pathogenic infections, and autoimmune disorders.

In that particular example, the antibody-drug linkage is limited to a disulfide or a thioether bond, which in general will likely lead to the heterogeneity and inconsistency problem described above. And there is few control, if any, about the attachment of multiple drugs. The instant invention allows multiple non-natural amino acids with different chemistry to be incorporated at different pre-determined positions of the antibody or its fragment, thus allowing multiple drug molecules to be site-specifically attached to the immunoconjugate.

Thus the invention provides an immunoconjugate comprising an antibody (or its functional fragment) specific for a target (e.g., a target cell), said antibody (or fragment or functional equivalent thereof) conjugated, at specific, pre-determined positions, with two or more therapeutic molecules, wherein each of said positions comprise a non-natural amino acid. In certain embodiments, the antibody fragments are F(ab′)₂, Fab′, Fab, or Fv fragments.

In certain embodiments, the two or more therapeutic molecules are the same. In certain embodiments, the two or more therapeutic molecules are different. In certain embodiments, the therapeutic molecules are conjugated to the same non-natural amino acids. In certain embodiments, the therapeutic molecules are conjugated to different non-natural amino acids.

In certain embodiments, the nature or chemistry of the non-natural amino acid/therapeutic molecule linkage allows cleavage of the linkage under certain conditions, such as mild or weak acidic conditions (e.g., about pH 4-6, preferably about pH5), reductive environment (e.g., the presence of a reducing agent), or divalent cations, and is optionally accelerated by heat. See EP0318948A2.

In certain embodiments, the non-natural amino acid(s) and/or the therapeutic molecule comprises a chemically reactive moiety. The moiety may be strongly electrophilic or nucleophilic and thereby be available for reacting directly with the therapeutic molecule or the antibody or fragment thereof. Alternatively, the moiety may be a weaker electrophile or nucleophile and therefore require activation prior to the conjugation with the therapeutic molecule or the antibody or fragment thereof. This alternative would be desirable where it is necessary to delay activation of the chemically reactive moiety until an agent is added to the molecule in order to prevent the reaction of the agent with the moiety. In either scenario, the moiety is chemically reactive, the scenarios differ (in the reacting with antibody scenario) by whether following addition of an agent, the moiety is reacted directly with an antibody or fragment thereof or is reacted first with one or more chemicals to render the moiety capable of reacting with an antibody or fragment thereof. In certain embodiments, the chemically reactive moiety includes an amino group, a sulfhydryl group, a hydroxyl group, a carbonyl-containing group, or an alkyl leaving group.

In certain embodiments, the therapeutic molecule is conjugated to the antibody through a linker/spacer (e.g., one or more repeats of methylene (—CH₂—), methyleneoxy (—CH₂—O—), methylenecarbonyl (—CH₂—CO—), amino acids, or combinations thereof).

Therapeutic molecules may include drugs, toxins (e.g. icin, abrin, diptheria toxin, and Pseudomonas exotoxin A), biological response modifiers, radiodiagnostic compounds, radiotherapeutic compounds, and derivatives or combinations thereof.

The therapeutic molecules may be linked to the antibody via a dissociable means, such as a cleavable covalent bond, an acid labile bond (such as hydrozone), or a non-covalent association stable at physiological environments, but unstable/dissociable under one or more pathological conditions, such as low pH and/or hypoxia. If the covalent bond is cleavable, it is preferably cleavable by an enzyme specifically found or selectively enriched at the pathological tissue (e.g. tumor site, etc.)

The invention also provides the use of the subject translation systems, host cells, and methods for generating such immunoconjugates.

C. pH-Sensitive Binding

Solid tumors typically have a lower pH compared to that of blood and other normal tissues. Similar to the generation of cytokines with enhanced half-life, and by using non-natural amino acids, one can produce antibodies that will differentiate between an antigen present on tumors cells, in a relatively low pH environment, and the same antigen present on non-tumor cells (healthy cells or circulating antigens), in a relatively high pH environment. Such an antibody will have improved binding affinity for the antigen at the tumor site by taking advantage of pH differences of the tumor environments.

In fact, many other pathological conditions are associated with local acidic environment. For example, tissue acidosis, a shift in tissue pH in the acidic direction, is a dominant factor in many pathophysiological states, and contributes largely to pain and hyperalgesia. Indeed, extracellular pH can drop from around pH=7.4 in non-pathological conditions to as low as pH=5.0 during inflammation, ischemia, infection, around tumors, and fractures, in hematomas, edema and blisters (Reeh and Steen, Prog. Brain Res., 113: 143, 1996; Helmlinger et al., Nat. Med., 3: 177, 1997; Clarke et al., NMR Biomed., 6: 278, 1993). Bicarbonate injections in abscesses were even used to relief from pain, as mentioned by Clarke et al. (supra). Tuberculosis abscess, a destructive inflammation state, produces an exudate of normal pH and is painless (see Clarke et al., supra). Local acidosis is thus a common feature of many painful states.

Inflammatory exudates (e.g. at infection site or around a tumor) and synovial fluid of arthritic joints are acidic. This is due to stimulated cells and immigrating leukocytes by important acid degranulation, the lysed cells, which liberate their acidic content, acids released by metabolism modification and by infectious agents when present. In ischemic muscle or heart (due to a high activity or arterial occlusion), acidosis results from lactic acid production due to the lack of oxygen, CO₂ retention due to impaired blood flow, and ATP breakdown, and this acidosis is worsened by intensive exercise. Disruption of the mucosal barrier of the gastro-intestinal tract, as observed in ulcers, or damage to the urinary tract epithelium, as in cystitis, exposes the underlying tissue to the low pH of gastric juice or urine respectively. The low pH observed in all these conditions is a strong contributor to pain and hypersensitivity.

Thus any protein-based therapies targeting these pathological conditions may benefit from the method of the instant invention. Any protein therapies for such pathological conditions may be modified by the subject non-natural amino acids, such that interaction with their respective targets may be modulated based on the target site pH.

In addition, pH sensitive binding can also be applied to other non-antibody molecules (such as IL-2, interferons, etc.) that are administered for solid tumor therapies.

### VIII. Exemplary Antibodies

Any antibodies, or their functional fragments or derivatives can be modified according to the instant invention.

In general, antibodies to a tumor antigen can be selected by a variety of techniques known to one of skill in the art. See, for example, Monoclonal antibodies: preparation and use of monoclonal antibodies and engineered antibody derivatives. Edited by Heddy Zola, Oxford: BIOS; New York Springer, c2000 (incorporated herein by reference).

Many antibodies to a variety of tumor antigens are known to one of skill in the art. For example, there are many FDA-approved and commercially marketed antibodies including (but are not limited to): RITUXAN® (Rituximab), TIUXAN (Ibritumomab), BEXXAR® (Tositumomab and Iodine I¹³¹ Tositumomab), HERCEPTIN® (Trastuzumab), ZEVALIN® (Ibritumomab Tiuxetan), AVASTIN™ (Bevacizumab), ERBITUX™ (Cetuximab), MYLOTARG™ (Gemtuzumab-Ozogamicin for Injection), CAMPATH™ (Alemtuzumab), PANOREX® (Edrecolomab), ZENAPAX® (Daclizumab), CeaVac (Anti-Idiotype (Anti-Id) Monoclonal Antibody (Mab)), IGN101 (murine mAb 17-1A), IGN311 (humanized monoclonal antibody), BEC2 (anti-idiotypic monoclonal antibody), IMC-1C11 (KDR receptor monoclonal antibody), LymphoCycle (Epratuzumab), or Pentumomab. All these antibodies may be modified using the instant methods to incorporate non-natural amino acid(s), thereby acquiring enhanced specificity/selectivity for tumor sites.

The following part describes one of these antibodies for illustration purpose only. These examples are by no means limiting in any respect. More detailed information regarding these commercially available/marketed antibodies may be obtained from the respective manufactures.

HERCEPTIN® (Trastuzumab)

Breast cancer is the most common malignancy among women in the United States, with 211,300 new cases projected for 2003 (Jemal et al., CA Cancer J. Clin. 53: 5-26, 2003). Amplification of the human epidermal growth factor receptor 2 (HER2) gene results in HER2 protein overexpression in approximately 25% of breast cancer patients (Slamon et al., Science 244: 707-712, 1989). The HER2 proto-oncogene encodes the production of a 185 kDa cell surface receptor protein known as the HER2 protein or receptor (Hynes and Stern, Biochim Biophys Acta. 1198(2-3): 165-184, 1994). This gene has homology to the rat gene neu, and is therefore sometimes referred to as HER2/neu or c-erbB-2. Normal cells express a small amount of HER2 protein on their plasma membranes in a tissue-specific pattern. In tumor cells, gene amplification of the HER2 gene may lead to an overexpression of HER2 protein, resulting in increased cell division and a higher rate of cell growth. HER2 gene amplification may also be associated with transformation to the cancer cell phenotype (Hynes and Stern, Biochim Biophys Acta. 1198(2-3): 165-184, 1994; Sundaresan et al., Curr Oncol Rep. 1: 16-22, 1999).

Unlike any conventional breast cancer chemotherapy or hormonal treatment, Herceptin® (Trastuzumab) monoclonal antibody therapy offers a unique therapeutic approach through unique mechanisms of action. Herceptin specifically targets the persistent, aggressive nature of HER2-driven metastatic breast cancer. Its proposed mechanisms of action include: potentiation of chemotherapy (cytotoxic), inhibition of tumor cell proliferation (cytostatic), and facilitation of immune function (cytotoxic).

### IX. General Techniques

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). These texts describe mutagenesis, the use of vectors, promoters and many other relevant topics related to, e.g., the generation of orthogonal tRNA, orthogonal synthetases, and pairs thereof.

Various types of mutagenesis are used in the present invention, e.g., to produce novel sythetases or tRNAs. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like.

The above texts and examples found herein describe these procedures as well as the following publications and references cited within: Sieber, et al., Nature Biotechnology, 19:456-460 (2001); Ling et al., Approaches to DNA mutagenesis: an overview, Anal Biochem. 254(2): 157-178 (1997); Dale et al., Oligonucleotide-directed random mutagenesis using the phosphorothioate method, Methods Mol. Biol. 57:369-374 (1996); I. A. Lorimer, I. Pastan, Nucleic Acids Res. 23, 3067-8 (1995); W. P. C. Stemmer, Nature 370, 389-91 (1994); Arnold, Protein engineering for unusual environments, Current Opinion in Biotechnology 4:450-455 (1993); Bass et al., Mutant Trp repressors with new DNA-binding specificities, Science 242:240-245 (1988); Fritz et al., Oligonucleotide-directed construction of mutations: a gapped duplex DNA procedure without enzymatic reactions in vitro, Nucl. Acids Res. 16: 6987-6999 (1988); Kramer et al., Improved enzymatic in vitro reactions in the gapped duplex DNA approach to oligonucleotide-directed construction of mutations, Nucl. Acids Res. 16: 7207 (1988); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Carter, Improved oligonucleotide-directed mutagenesis using M13 vectors, Methods in Enzymol. 154: 382-403 (1987); Kramer & Fritz Oligonucleotide-directed construction of mutations via gapped duplex DNA, Methods in Enzymol. 154:350-367 (1987); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel et al., Rapid and efficient site-specific mutagenesis without phenotypic selection, Methods in Enzymol. 154, 367-382 (1987); Zoller & Smith, Oligonucleotide-directed mutagenesis: a simple method using two oligonucleotide primers and a single-stranded DNA template, Methods in Enzymol. 154:329-350 (1987); Carter, Site-directed mutagenesis, Biochem. J. 237:1-7 (1986); Eghtedarzadeh & Henikoff, Use of oligonucleotides to generate large deletions, Nucl. Acids Res. 14: 5115 (1986); Mandecki, Oligonucleotide-directed double-strand break repair in plasmids of Escherichia coli: a method for site-specific mutagenesis, Proc. Natl. Acad. Sci. USA, 83:7177-7181 (1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Wells et al., Importance of hydrogen-bond formation in stabilizing the transition state of subtilisin, Phil. Trans. R. Soc. Lond. A 317: 415-423 (1986); Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Grundström et al., Oligonucleotide-directed mutagenesis by microscale ‘shot-gun’ gene synthesis, Nucl. Acids Res. 13: 3305-3316 (1985); Kunkel, Rapid and efficient site-specific mutagenesis without phenotypic selection, Proc. Natl. Acad. Sci. USA 82:488-492 (1985); Smith, In vitro mutagenesis, Ann. Rev. Genet. 19:423-462 (1985); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Cassette mutagenesis: an efficient method for generation of multiple mutations at defined sites, Gene 34:315-323 (1985); Kramer et al., The gapped duplex DNA approach to oligonucleotide-directed mutation construction, Nucl. Acids Res. 12: 9441-9456 (1984); Kramer et al., Point Mismatch Repair, Cell 38:879-887 (1984); Nambiar et al., Total synthesis and cloning of a gene coding for the ribonuclease S protein, Science 223: 1299-1301 (1984); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis using M13-derived vectors: an efficient and general procedure for the production of point mutations in any DNA fragment, Nucleic Acids Res. 10:6487-6500 (1982). Additional details on many of the above methods can be found in Methods in Enzymology Volume 154, which also describes useful controls for trouble-shooting problems with various mutagenesis methods.

Oligonucleotides, e.g., for use in mutagenesis of the present invention, e.g., mutating libraries of synthetases, or altering tRNAs, are typically synthesized chemically according to the solid phase phosphoramidite triester method described by Beaucage and Caruthers, Tetrahedron Letts. 22(20):1859-1862, (1981) e.g., using an automated synthesizer, as described in Needham-VanDevanter et al., Nucleic Acids Res., 12:6159-6168 (1984).

In addition, essentially any nucleic acid can be custom or standard ordered from any of a variety of commercial sources, such as The Midland Certified Reagent Company, The Great American Gene Company, ExpressGen Inc., Operon Technologies Inc. (Alameda, Calif.) and many others.

The present invention also relates to host cells and organisms for the in vivo incorporation of a non-natural amino acid via orthogonal tRNA/RS pairs. Host cells are genetically engineered (e.g., transformed, transduced or transfected) with the vectors of this invention, which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation (From et al., Proc. Natl. Acad. Sci. USA 82, 5824 (1985), infection by viral vectors, high velocity ballistic penetration by small particles with the nucleic acid either within the matrix of small beads or particles, or on the surface (Klein et al., Nature 327, 70-73 (1987)). Berger, Sambrook, and Ausubel provide a variety of appropriate transformation methods.

The engineered host cells can be cultured in conventional nutrient media modified as appropriate for such activities as, for example, screening steps, activating promoters or selecting transformants. These cells can optionally be cultured into transgenic organisms.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

Several well-known methods of introducing target nucleic acids into bacterial cells are available, any of which can be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria, (see, e.g., EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (all supra). A catalogue of Bacteria and Bacteriophages useful for cloning is provided, e.g., by the ATCC, e.g. The ATCC Catalogue of Bacteria and Bacteriophage (1992) Gherna et al. (eds.) published by the ATCC. Additional basic procedures for sequencing, cloning and other aspects of molecular biology and underlying theoretical considerations are also found in Watson et al. (1992) Recombinant DNA Second Edition Scientific American Books, NY.

### EXAMPLES

This invention is further illustrated by the following examples which should not be construed as limiting. The teachings of all references, patents and published patent applications cited throughout this application, as well as the Figures are hereby incorporated by reference.

Examples I-III illustrate the general method of site-specific incorporation of non-natural amino acid using the degenerate codon orthogonal system. Example IV illustrates substitution of natural amino acids with non-natural amino acids to alter pH-sensitive binding in one representative protein—the HERCEPTIN monoclonal antibody.

### Example I tRNA and Synthetase Construction

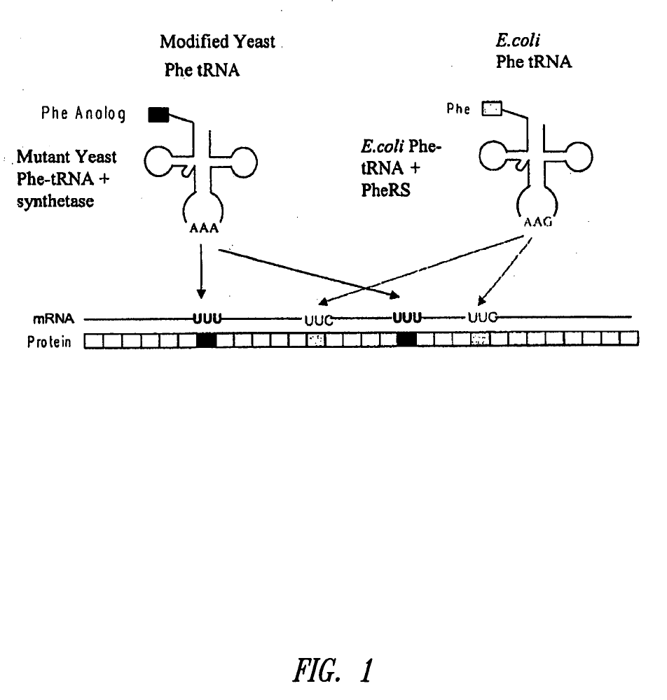

This example illustrates the incorporation of an amino acid analog in proteins at positions encoded by codons which normally encode phenylalanine (Phe). A schematic diagram is shown in FIG. 1. Similar approaches can be used for any other analogs.

Phe is encoded by two codons, UUC and UUU. Both codons are read by a single tRNA, which is equipped with the anticodon sequence GAA. The UUC codon is therefore recognized through standard Watson-Crick base-pairing between codon and anticodon; UUU is read through a G-U wobble base-pair at the first position of the anticodon (Crick, J. Mol. Biol. 19: 548, 1966; Soll and RajBhandary, J. Mol. Biol. 29: 113, 1967). Thermal denaturation of RNA duplexes has yielded estimates of the Gibbs free energies of melting of G-U, G-C, A-U, and A-C basepairs as 4.1, 6.5, 6.3, and 2.6 kcal/mol, respectively, at 37° C. Thus the wobble basepair, G-U, is less stable than the Watson-Crick basepair, A-U. A modified tRNA^{Phe} outfitted with the AAA anticodon (tRNA^{Phe}_{AAA}) was engineered to read the UUU codon, and was predicted to read such codons faster than wild-type tRNA^{Phe}_{GAA}. See FIG. 1.

Although tRNAs bearing unmodified A in the first position of the anticodon are known to read codons ending with C or U (Inagaki et al., J. Mol. Biol. 251: 486, 1995; Chen et al., J. Mol. Biol. 317: 481, 2002; Boren et al., J. Mol. Biol. 230: 739, 1993), the binding of E. coli tRNA^{Phe}_{GAA} at UUC should dominate that of tRNA^{Phe}_{AAA}, owing to differences in the stability of A-C and G-C base pairs (see above).

We prepared a modified yeast tRNA^{Phe} (ytRNA^{Phe}_{AAA}) with an altered anticodon loop. The first base (G34) of the tRNA^{Phe}_{GAA} was replaced with A to provide specific Watson-Crick base-pairing to the UUU codon. Furthermore, G37 in the extended anticodon site was replaced with A to increase translational efficiency (see Furter, Protein Sci. 7: 419, 1998). We believe that charging of ytRNA^{Phe}_{AAA} by E. coli PheRS can be ignored, because the aminoacylation rate of ytRNA^{Phe}_{AAA} by E. coli PheRS is known to be <0.1% of that of E. coli tRNA^{Phe}_{GAA} (Peterson and Uhlenbeck, Biochemistry 31: 10380, 1992).

Since wild-type yeast PheRS does not activate amino acids significantly larger than phenylalanine, a modified form of the synthetase with relaxed substrate specificity was prepared to accommodate L-3-(2-naphthyl)alanine (NaI).

The modified yeast PheRS (mu-yPheRS) was prepared by introduction of a Thr415Gly mutation in the α-subunit of the synthetase (Datta et al., J. Am. Chem. Soc. 124: 5652, 2002). The kinetics of activation of NaI and Phe by mu-yPheRS were analyzed in vitro via the pyrophosphate exchange assay. The specificity constant (k_{cat}/K_{M}) for activation of NaI by mu-yPheRS was found to be 1.55×10⁻³ (s⁻¹M⁻¹), 8-fold larger than that for Phe. Therefore, when the ratio of NaI to Phe in the culture medium is high, ytRNA^{Phe}_{AAA} should be charged predominantly with NaI.

### Example II Generation of a Mutant Protein Containing NaI

Murine dihydrofolate reductase (mDHFR), which contains nine Phe residues, was chosen as the test protein. The expression plasmid pQE16 encodes mDHFR under control of a bacteriophage T5 promoter; the protein is outfitted with a C-terminal hexahistidine (HIS₆) tag to facilitate purification via immobilized metal affinity chromatography.

In this construct, four of the Phe residues of mDHFR are encoded by UUC codons, five by UUU. A full-length copy of the mu-yPheRS gene, under control of a constitutive tac promoter, was inserted into pQE16. The gene encoding ytRNA^{Phe}_{AAA} was inserted into the repressor plasmid pREP4 (Qiagen) under control of the constitutive promoter lpp. E. coli transformants harboring these two plasmids were incubated in Phe-depleted minimal medium supplemented with 3 mM NaI and were then treated with 1 mM IPTG to induce expression of mDHFR. Although the E. coli strain (K10-F6) used in this study is a Phe auxotroph, (see Furter, supra) a detectable level of mDHFR was expressed even under conditions of nominal depletion of Phe, probably because of release of Phe through turnover of cellular proteins. In negative control experiments, mDHFR was expressed in the absence of either ytRNA^{Phe}_{AAA} or mu-yPheRS. The molar mass of mDHFR prepared in the absence of NaI, ytRNA^{Phe}_{AAA}, or mu-yPheRS was 23,287 Da, precisely that calculated for HIS-tagged mDHFR. However, when ytRNA^{Phe}_{AAA} and mu-yPheRS were introduced into the expression strain and NaI was added to the culture medium, the observed mass of mDHFR was 23,537 Da (yield 2.5 mg/L after Ni-affinity chromatography). Because each substitution of NaI for Phe leads to a mass increment of 50 Da, this result is consistent with replacement of five Phe residues by NaI. No detectable mass shift was found in the absence of either ytRNA^{Phe}_{AAA} or mu-yPheRS, confirming that the intact heterologous pair is required for incorporation of NaI. For mDHFR isolated from the strain harboring the heterologous pair, amino acid analysis indicated replacement of 4.4 of the 9 Phe residues by NaI. Without ytRNA^{Phe}_{AAA} or mu-yPheRS, no incorporation of NaI into mDHFR was detected by amino acid analysis.

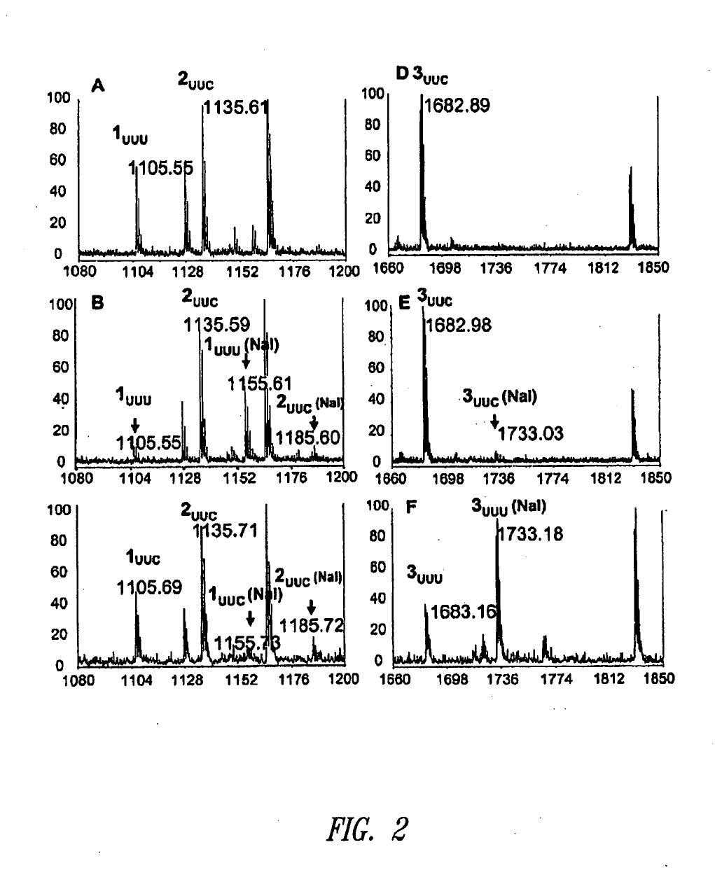

Tryptic digests of mDHFR were analyzed to determine the occupancy of individual Phe sites. Digestion of mDHFR yields peptide fragments that are readily analyzed by MALDI mass spectrometry as shown in FIG. 2. Peptide uuu (residues 184-191, YKFEVYEK, SEQ ID NO: 1) contains a Phe residue encoded as UUU, whereas peptides 2_{UUC} (residues 62-70, KTWFSIPEK, SEQ ID NO: 2) and 3_{UUC} (residues 26-39, NGDLPWPPLRNEFK, SEQ ID NO: 3) each contain a Phe residue encoded as UUC. In the absence of NaI, peptide 1_{UUU} was detected with a monoisotopic mass of 1105.55 Da, in accord with its theoretical mass (FIG. 2A). However, when NaI was added, a strong signal at a mass of 1155.61 Da was detected, and the 1105.55 was greatly reduced in intensity (FIG. 2B). As described earlier, each substitution of NaI for Phe leads to a mass increase of 50.06 Da; the observed shift in mass is thus consistent with replacement of Phe by NaI in response to the UUU codon. Liquid chromatography—tandem mass spectrometry (LC/MS/MS) confirmed this assignment. The ratio of MALDI signal intensities, though not rigorously related to relative peptide concentrations, suggests that NaI incorporation is dominant at the UUU codon.

Similar analyses were conducted for peptides 2_{UUC} and 3_{UUC}. In the absence of added NaI, the observed masses of peptides 2_{UUC} and 3_{UUC} are 1135.61 (FIG. 2A) and 1682.89 Da (FIG. 2D), respectively, as expected. Upon addition of NaI to the expression medium, the 1135.61 signal and 1682.89 signals were not substantially reduced, and only weak signals were observed at masses of 1185.60 and 1733.03 (FIGS. 2B and 2E), which would be expected for peptides 2_{UUC} and 3_{UUC} containing NaI. NaI incorporation thus appears to be rare at UUC codons under the conditions used here for protein expression.

There is at least a formal possibility that the observed codon-biased incorporation of NaI might be dependent on codon context rather than, or in addition to, codon identity. MALDI sampling errors are also possible. To test these possibilities, a mutant mDHFR gene was prepared by mutating the UUU codon in peptide 1_{UUU} to UUC, and the UUC codon in peptide 3_{UUC} to UUU. In the resulting peptide 1_{UUC}, the signal indicating incorporation of NaI was only slightly above background (FIG. 2C), whereas for peptide 3_{UUU}, NaI is readily detected (FIG. 2F). NaI incorporation is unambiguously codon-biased to UUU.

The results described here show conclusively that a heterologous pair comprising a genetically engineered tRNA and cognate aminoacyl-tRNA synthetase can be used to break the degeneracy of the genetic code in E. coli.

### Example III Application to Degenerate Leucine-Encoding Codons

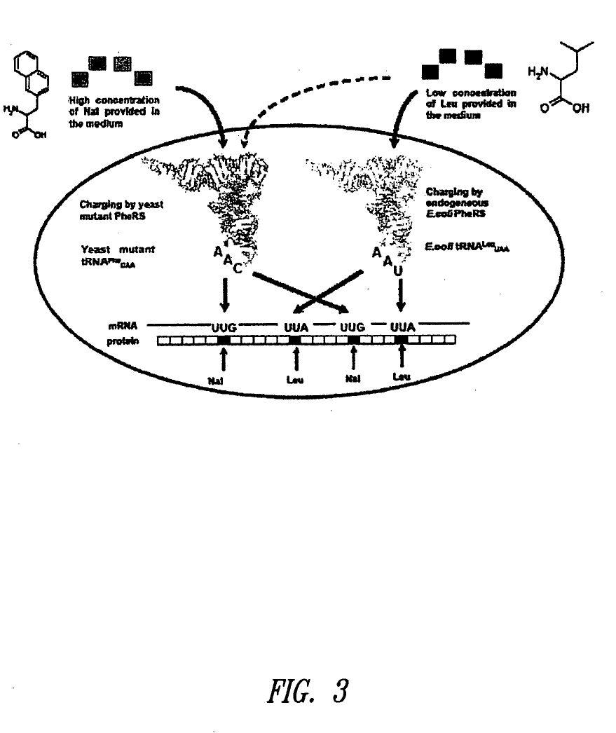

In this example, multiple-site-specific incorporation of a non-natural amino acid into murine dihydrofolate reductase (mDHFR) in response to a sense codon was realized by use of an E. coli strain outfitted with a yeast transfer RNA (ytRNA^{phe}_{CAA}) capable of Watson-Crick base-pairing with the leucine (Leu) codon UUG. ytRNA^{phe}_{CAA} was charged with L-3-(2-naphthyl)alanine (NaI) by a co-expressed modified yeast phenylalanine tRNA synthetase. See schematic diagram in FIG. 3. Mass spectrometric analysis of tryptic digests of mDHFR showed that the UUG codon was partially re-assigned to NaI, whereas the other five Leu codons remained assigned to Leu.

Incomplete occupancy of the UUG codon by NaI is due at least in part to competition with leucine-charged E. coli tRNA^{Leu}s. In an attempt to reduce competition by E. coli tRNA^{Leu}s, use of a mutant E. coli strain lacking tRNA^{Leu}_{CAA} and addition of an E. coli leucyl-tRNA synthetase (LeuRS) inhibitor were tested. A Phe/Leu double auxotrophic strain derived from the tRNA^{Leu}_{CAA}-deficient strain XA106 (CGSC at Yale) was tested for incorporation of NaI at the UUG codon. Introduction of ytRNA^{Phe}_{CAA} into a mutant host lacking tRNA^{Leu}_{CAA} did not enhance the occupancy of the UUG sites by NaI, consistent with earlier proposals that E. coli tRNA^{Leu}_{CAA} is rarely involved in protein translation (Holmes, W. M.; Goldman, E.; Miner, T. A.; Hatfield, G. W. Proc. Natl. Acad. Sci. USA 74: 1393-1397, 1977). 4-Aza-DL-leucine (AZL) is a competitive inhibitor of E. coli LeuRS, and does not progress to the azaleucyl-adenylate in vitro. Although addition of AZL reduced the growth rate of the host due to reduced activation of Leu by E. coli LeuRS, it resulted in enhanced occupancy of the LUG codon by NaI. The results described here demonstrate conclusively that the concept of breaking the degeneracy of the genetic code is quite general.

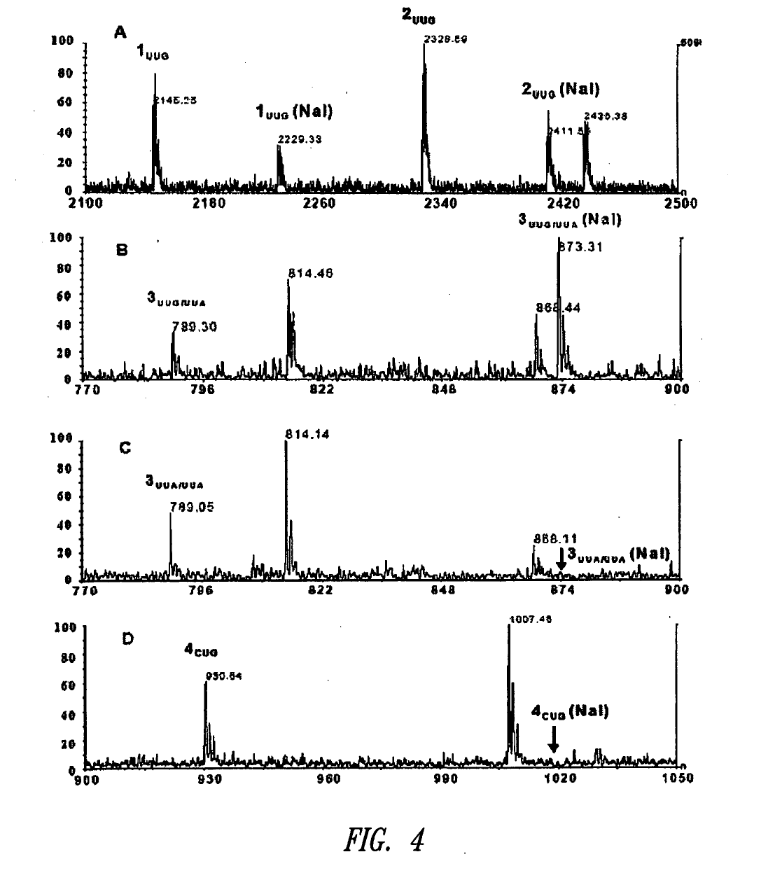

Replacement of Leu by NaI was detected in MALDI mass spectra of tryptic fragments of mDHFR (FIG. 4). Peptide 1_{UUG} (residues 145-162, IMQEFESDTFFPEIDL_{UUG}GK, SEQ ID NO: 4) contains a Leu residue encoded by UUG, whereas Peptide 1_{UUG} (NaI) refers to the form of the peptide containing NaI in place of Leu. Peptides 2_{UUG} (residues 3-25, GSGIMRPL_{UUG}NSIVAVSQNMGIGK, SEQ ID NO: 5), and 4_{CUG} (residues 54-61, QNL_{CUG}VIMGR, SEQ ID NO: 6) were designated similarly. Peptide 3_{UUG/UUA} (residues 99-105, SL_{UUG}DDAL_{UUA}R, SEQ ID NO: 7) contains two Leu residues encoded as UUG and UUA, respectively, while Peptide 3_{UUA/UUA} contains two Leu residues encoded as only UUA. Upon addition of Nal, the masses of peptide fragments 1-3 shift by 84.06 (1_{UUG}), 83.89 (2_{UUG}), and 84.18 (3_{UUG/UUA}) mass units, respectively, as expected for replacement of Leu by the larger Phe analog (NaI). The tandem mass spectrum of Peptide 3_{UUG/UUA} (NaI) confirmed that only the Leu encoded by UUG was replaced by NaI. Furthermore, NaI incorporation was not detected when UUG was mutated to UUA in Peptide 3. No signal corresponding to Peptide 4_{CUG} (NaI) was detected, whereas that corresponding to Peptide 4_{CUG} was detected at 904.54 mass units. These data confirm that incorporation of NaI is strongly biased to UUG.

Replacement of Leu by NaI was detected in MALDI mass spectra of tryptic fragments of mDHFR expressed in tRNA^{Leu}_{CAA}-harboring E. coli (a) and tRNA^{Leu}_{CAA}-deficient E. coli (b). Peptide 3_{UUG/UUA} (residues 99-105, SL_{UUG}DDAL_{UUA}R, SEQ ID NO: 7) contains two Leu residues encoded as UUG and UUA, respectively. Upon addition of NaI, the masses of these fragments shift in accord with the mass difference between NaI and Leu, indicating that incorporation had occurred.

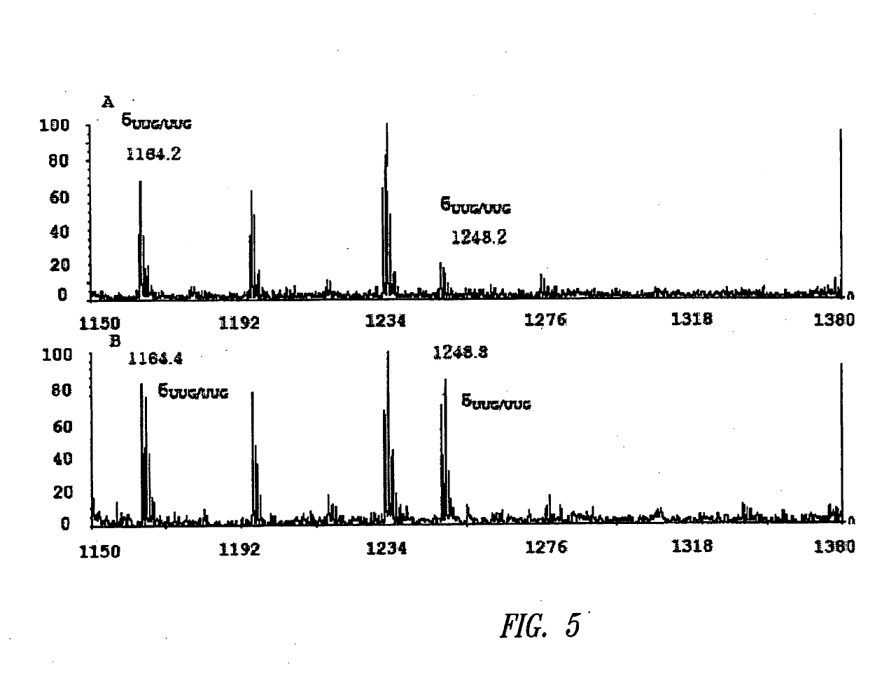

FIG. 5 shows the effect of AZL on replacement of Leu by NaI was evaluated by MALDI mass spectra of tryptic fragments of mDHFR. Peptide 5_{UUG/UUG} (residues 26-35, NGDL_{UUG}PWPPL_{UUG}R, SEQ ID NO: 8) contains two Leu residues encoded as UUG. Upon addition of NaI, the masses of these fragments shift in accord with the mass difference between NaI and Leu. Only NaI (a), NaI and 1 mM AZL (b) were supplemented into the media.

### Example IV HER2-Neu Tumor-Specific Antibodies

Herceptin® (Trastuzumab) is a monoclonal antibody against breast cancer but has a serious side effect that it affects the hearts of the patients. This is partly because Herceptin® (Trastuzumab) binds to the her2 receptor, which is expressed on both tumor cells and normal heart cells. Thus a conditionally tumor-specific Herceptin® or its functional fragments would be very useful in reducing these side effects.

According to the methods of the invention, certain histidines in Herceptin® Fab, Scfv, or other functional fragments are replaced with variants such as triazoles and other moieties that have a lower pKa. The histidines are modified both site-specifically and non-site specifically. New sites are added at the binding interface for pH-sensitive histidine analogs.

Sequences of Herceptin® Fab is listed below.

### Light Chain—Human Her2 Chain A has 214 Amino Acids:

(SEQ ID NO: 9)

DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Heavy Chain—Chain B has 220 Amino Acids:

(SEQ ID NO: 10)

EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEP

The histidines that are replaced by non-natural amino acids are underlined. The doubly underlined histidines are present in the variable domains and the singly underlined histidines are in the constant domains. In addition to the existing histidines, other non-histidine residues, especially those not essential for maintaining the structures and/or functions of the antibody, are modified to histidine codons to enable the incorporation of histidine analogs at those sites. In cases of incorporating multiple histidine analogs, the incorporated histidine analogs may be either the same or different.

### Example V Exemplary Histidine Analogs

For illustration purpose only, this example provides an example of designing certain non-natural amino acid analogs of histidine.

We used quantum mechanical calculations to derive the acid dissociation constants (pKa) of a series of histidine derivatives using Jaguar™ 5.5 software (Schrödinger, LLC, June 2004. User Manual downloadable from the Schrödinger website, and incorporated herein by reference). Specifically, we substituted the hydrogens at the 2, 4, or both positions of the histidine imidazole ring with the functional groups listed in the table X below. For this experiment, these functional groups were selected because they are relatively small in size and lack the ability to form strong hydrogen bonds that could affect protein binding.

Our calculations suggest that these groups shift the histidine pKa (6.0 in experimental measurement and 5.8 in our calculation) upward or downward depending on their electron donating or withdrawing capabilities. The table lists the calculated pKa values of non-natural histidines that can be used in various applications to control pH-responsive protein binding. It is apparent that, even with the limited choice of just 6 types of small side-chain groups (e.g. —CN, —F, —Cl, —CH₂F, —OCH₃, or —CH₃), and two potential substitution positions (position 2 or 4 on the imidazole ring) of a single natural amino acid (e.g. histidine), the side-chain pKa of the resulting histidine analog can range from a low of −8.4 to a high of 8.2 (an enormous difference of more than 1016), with 15 different values in between. A combination of different small side-chain groups at different ring positions is expected to create more pKa values.

Compared with a single value (pKa=5.8) of the natural histidine residue, this range greatly expanded the possibility of modulating pH-sensitive binding of a protein bearing such a non-natural amino acid.

TABLE X
pKa for Exemplary Histidine Analogs

| | Position 2 | Position 4 | Position 2, 4 |
|---|---|---|---|
| CN | −1.2 | −0.6 | −8.4 |
| F | 0.9 | 0.4 | −4.1 |
| Cl | 1.4 | 1.2 | −2.8 |
| CH2F | 4.1 | 4.4 | 2.4 |
| OCH3 | 4.9 | 3.5 | 3.5 |
| H | 5.8* | 5.8 | 5.8 |
| CH3 | 7.2 | 6.9 | 8.2 |

Next, we substituted the histidine at the center of a leucine zipper c-MYC-MAX heterodimer with an unnatural histidine where its hydrogen atom at the 2 position of the imidazole ring was substituted with a methyl group (see the last line of Table X). After we incorporated the non-natural histidine, we sampled the histidine rotamer library to calculate the energy of each conformation. We found that the lowest energy rotamer for 2-methyl histidine has the same conformation as the wild-type histidine in the NMR structure. The quantum mechanical pKa calculations suggest that the substitution will shift the histidine pKa (7.19 experimental value) up for 1.4 units in the particular context of the leucine zipper. This indicates that more variations in side-chain pKa values may be created in different target protein contexts, further increasing the potential pKa choices.

Lastly, we observed that the 2-methyl histidine and the widetype histidine have a similar effect on target protein binding in the zipper. However, the effect was achieved at different pH values due to the side-chain pKa differences, which illustrates and conforms with our design purpose.

The contents of all cited references (including literature references, issued patents, published patent applications as cited throughout this application) are hereby expressly incorporated by reference.

### EQUIVALENTS

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

1. A modified protein comprising one or more non-natural amino acid(s), said non-natural amino acid(s) confers or substantially alters pH-sensitive binding of said protein to its binding partner.

2. The modified protein of claim 1, wherein said binding partner is a polypeptide, a nucleic acid, a polysaccharide, a lipid, a steroid, a polymer, a small molecule, or a metal ion.

3. The modified protein of claim 1, which is a modified antibody.

4. The modified protein of claim 3, wherein the non-natural amino acid(s) confers the modified antibody enhanced specifically, selectively, or affinity towards an antigen in a tissue at a specific pH.

5. The modified protein of claim 4, wherein said specific pH is an extracellular pH at least about 0.5 or about 1.0-1.5 units higher or lower than a physiological pH.

6. The modified protein of claim 4, wherein said tissue is a neoplastic tissue, such as breast cancer overexpressing HER-2/neu.

7. The modified protein of claim 4, wherein said tissue is undergoing a pathological condition selected from: tissue acidosis, inflammation, ischemia, infection, around tumors, fracture, hematoma, edema, blister, Tuberculosis abscess, a destructive inflammation state, arthritic, ulcer, or cystitis.

8. The modified protein of claim 4, which is a modified monoclonal antibody, or a functional fragment or derivative thereof selected from: Fab, Fab′, F(ab)₂, Fd, Fv, ScFv, diabody, tribody, tetrabody, dimer, trimer, or minibody.

9. The modified protein of claim 4, which is modified based on RITUXAN® (Rituximab), TIUXAN (Ibritumomab), BEXXAR® (Tositumomab and Iodine I¹³¹ Tositumomab), HERCEPTIN® (Trastuzumab), ZEVALIN® (Ibritumomab Tiuxetan), AVASTIN™ (Bevacizumab), ERBITUX™ (Cetuximab), MYLOTARG™ (Gemtuzumab-Ozogamicin for Injection), CAMPATH® (Alemtuzumab), PANOREX® (Edrecolomab), ZENAPAX® (Daclizumab), CeaVac (Anti-Idiotype (Anti-Id) Monoclonal Antibody (Mab)), IGN101 (murine mAb 17-1A), IGN311 (humanized monoclonal antibody), BEC2 (anti-idiotypic monoclonal antibody), IMC-1C11 (KDR receptor monoclonal antibody), LymphoCycle (Epratuzumab), or Pentumomab.

10. The modified protein of claim 4, which is modified by substituting one or more natural amino acid(s) in said antibody with said non-natural amino acid(s).

11. The modified protein of claim 10, wherein said natural amino acid(s) is histidine.

12. The modified protein of claim 10, wherein said non-natural amino acid(s) is selected from: 1,2,4-triazole-3-alanine, 2-fluoro-histidine, L-methyl histidine, 3-methyl-L-histidine, β-2-thienyl-L-alanine, or β-(2-Thiazolyl)-DL-alanine.

13. The modified protein of claim 10, wherein said non-natural amino acid is a histidine analog with one or more substitutions on positions 2 and 4 of the histidine imidazole ring, by one or more of the groups selected from: —CN, —F, —Cl, —CH₂F, —OCH₃, or —CH₃.

14. The modified protein of claim 10, wherein said natural amino acid(s) is present in the Fc-region, the Fab-region, the V_{H} region, or the binding interface of said antibody.

15. The modified protein of claim 14, wherein said non-natural amino acid(s) confer enhanced binding affinity to Fc-receptor and/or to Clq of the complement system.

16. The modified protein of claim 10, wherein said non-natural amino acid(s) is sterically similar or dissimilar to said natural amino acid(s).

17. The modified protein of claim 16, further comprising mutated amino acid(s) adjacent to said non-natural amino acid(s) for maintaining binding affinity and/or specificity of said antibody.

18. The modified protein of claim 10, wherein two or more natural amino acids in said antibody are substituted with at least two different non-natural amino acids.

19. The modified protein of claim 4, wherein the non-natural amino acid(s) does not substantially alter the affinity/specificity of said modified antibody for said antigen.

20. The modified protein of claim 4, which has an enhanced affinity for said antigen in a tumor environment compared to a non-tumor environment.

21. The modified protein of claim 20, wherein the non-natural amino acid(s) has a side-chain pKa between the pH at the tumor environment and the pH at the non-tumor environment.

22-31. (canceled)

32. The modified protein of claim 1, which is a modified protein ligand, and wherein said binding partner is a cell-surface receptor, wherein said protein ligand undergoes receptor-mediated endocytosis.

33. The modified protein of claim 32, which binds the cell-surface receptor at a first pH, and does not substantially bind the cell-surface receptor at a second pH.

34. The modified protein of claim 33, wherein the first and the second pH is at least about 0.5 pH unit apart, preferably about 1, 1.5, 2, 2.5, 3, 3.5, 4 or more pH units apart.

35. The modified protein of claim 33, wherein the binding constant between the protein ligand and the cell-surface receptor at the first pH is at least about twice, three times, five times, 10 times, 20 times, 30 times, 50 times, 100 times, or 1000 times lower than that at the second pH.

36. The modified protein of claim 33, wherein the first pH is the local extracellular pH of the protein ligand-cell surface receptor complex, and the second pH is endosomal pH.

37. The modified protein of claim 32, wherein the protein ligand is a toxin or lectin selected from: Diptheria Toxin, Pseudomonas toxin, Cholera toxin, Ricin, or Concanavalin A; a viruses selected from: Rous sarcoma virus, Semliki forest virus, Vesicular stomatitis virus, or Adenovirus; a serum transport protein selected from: Transferrin, Low density lipoprotein, Transcobalamin, or Yolk protein; an antibody selected from: IgE, Polymeric IgA, Maternal IgG, or IgG (via Fc receptors); or a hormone or a growth factor selected from: insulin, EGF, Growth Hormone, Thyroid stimulating hormone, NGF, Calcitonin, Glucagon, Prolactin, Luteinizing Hormone, Thyroid hormone, PDGF, Interferon, or Catecholamine.

38.-50. (canceled)
